(19) United States
(12) Patent Application Publication
(10) Pub. No.: US 2023/0329887 A1
(43) Pub. Date: Oct. 19, 2023

(54) MULTIPLE FLARE AND SHOULDER ANTI MIGRATION STENT

(71) Applicant: Boston Scientific Scimed Inc., Maple Grove, MN (US)

(72) Inventors: Paul E. Tierney, Athenry (IE); David Collins, Galway (IE); Darren Gerard Curran, Galway (IE); Rebecca Lenehan, Kilkelly (IE)

(73) Assignee: Boston Scientific Scimed Inc., Maple Grove, MN (US)

(21) Appl. No.: 18/135,943

(22) Filed: Apr. 18, 2023

Related U.S. Application Data
(60) Provisional application No. 63/331,997, filed on Apr. 18, 2022.

(51) Int. Cl. A61F 2/90; A61F 2/00

(52) U.S. Cl. CPC A61F 2/90; A61F 2/0077; A61F 2220/0008; A61F 2230/0069; A61F 2250/0039; A61F 2210/0076

(57) ABSTRACT

The present disclosure relates to systems, devices and procedures for forming flow paths within or across body lumens. In one example, a stent comprises an elongate body configured to expand between a first constrained configuration and a second unconstrained configuration. The elongate body in the second unconstrained configuration may include a first end, a second end, and a cylindrical saddle region extending therebetween. The first end may comprise a first retention member and a second retention member, and the second end may comprise a third retention member and a fourth retention member.

### CROSS-REFERENCE TO RELATED APPLICATIONS

This application claims the benefit of priority under 35 U.S.C. §119 of U.S. Provisional Application No. 63/331,997, filed Apr. 18, 2022, the entire disclosure of which is hereby incorporated by reference.

### FIELD

The present disclosure relates generally to the field of medical devices. In particular, the present disclosure relates to medical devices for facilitating the flow of fluids and materials within or across body lumens, such as a stent which maintains an open flow passage within, across or between body lumens.

### BACKGROUND

Placement of a self-expanding stent (e.g., self-expanding metal stent or SEMS) within an anatomical area (e.g., body lumen, passage, vessel, duct, etc.) may enable fluid communication from one area to another. For example, a stent may enable flow of material from one body lumen to another.

However, available stents may carry various disadvantages. For example, stents may be more likely to dislodge or migrate from a desired placement, such as in response to forces generated by a patient's motion or natural peristaltic motion of surrounding tissue.

Various available stents may be configured to resist migration via traumatic engagement of tissue, for example, via protrusions configured to latch into tissue, in some cases causing trauma to the tissue that may contribute to pain and/or infection risk for a patient.

Accordingly, there is a need for devices which may efficiently interface with tissue, inhibiting migration without substantially injuring the tissue. A variety of advantageous medical outcomes may be realized by the devices, systems and/or methods of the present disclosure.

### SUMMARY

In an aspect, a stent may comprise an elongate body configured to be expandable between a first constrained configuration and a second unconstrained configuration. The elongate body in the second unconstrained configuration may include a first end, a second end, and a cylindrical saddle region extending therebetween. The first end may comprise a first retention member and a second retention member. The second end may comprise a third retention member and a fourth retention member.

In the above and other aspects, the second retention member may comprise a substantially cylindrical first radially outward surface, the third retention member may comprise a substantially cylindrical second radially outward surface, or both. The second retention member may comprise a first axially inner face joined to the first radially outward surface at a first corner, the third retention member may comprise a second axially inner face joined to the second radially outward surface at a second corner, or both. The first axially inner face may comprise a curved profile, the second axially inner face may comprise a curved profile, or both. The first corner may comprise an interior angle of 90 degrees or less. The second corner may comprise an interior angle of 90 degrees or less. The second retention member may be uncovered along the radially outwardmost surface. The third retention member may be uncovered along the radially outwardmost surface. The first corner may be uncovered. The second corner may be uncovered. The first retention member may be configured to interface with tissue substantially along an entire first longitudinal length thereof. The fourth retention member may be configured to interface with tissue substantially along an entire second longitudinal length thereof. The first end may comprise a first cylindrical portion extending between the first retention member and the second retention member, the second end may comprise a second cylindrical portion extending between the third retention member and the fourth retention member, or both. The first and fourth retention members, the second and third retention members, or the first, second, third, and fourth retention members may each be configured to atraumatically interface with tissue about a radially outwardmost circumference of the respective retention member. The stent may be formed from at least one braided or woven filament comprising a different pitch density, angle, or pattern at one or more of the first retention member, the second retention member, the cylindrical saddle region, the third retention member, or the fourth retention member, or any combination thereof, in the second configuration. The first retention member may comprise an asymmetrical cross section in an orthogonal plane to a longitudinal axis extending through the lumen of the body, the second retention member may comprise an asymmetrical cross section in an orthogonal plane to the longitudinal axis, or both. The first retention member may comprise an asymmetrical cross section in a longitudinal plane parallel to a longitudinal axis extending through the lumen of the body, the second retention member may comprise an asymmetrical cross section in a longitudinal plane parallel to the longitudinal axis, or both. The elongate body may comprise at least a partial cover.

In another aspect, a medical device may comprise an elongate body defining a lumen extending therethrough. The elongate body may be configured to transition between a first configuration and a second configuration. In the second configuration, the elongate body may comprise at least four retention features. The at least four retention features may comprise at least second and third retention members each comprising a shoulder. One or more shoulders of the at least first and second retention members may each comprise an interior angle of 90 angles or less. One or more shoulders may be configured to interface with tissue. The at least four retention members may comprise at least first and fourth retention members configured to atraumatically interface with tissue. In the second configuration, the elongate body may comprise a cylindrical region extending between the second and third retention members, between the first and second retention members, between the third and fourth retention members, or any combination thereof.

In the above and other aspects, one or more of the first, second, third, or fourth retention members may be configured to atraumatically interface with tissue about a radially outwardmost circumference of the respective retention member. The first retention member may comprise an asymmetrical cross section in an orthogonal plane to a longitudinal axis extending through the lumen of the body. The second retention member may comprise an asymmetrical cross section in an orthogonal plane to the longitudinal axis. The elongate body may be formed from at least one braided or woven filament. The at least one braided or woven filament may comprise a different pitch density, angle, or pattern at the first retention member, the second retention member, the cylindrical saddle region, the third retention member, or the fourth retention member, or any combination thereof, in the second configuration. The elongate body may comprise at least a partial cover.

In another aspect, a system may comprise a delivery catheter. The delivery catheter may comprise an inner member. The delivery catheter may comprise an outer sheath slidably disposed about the inner member. The delivery catheter may comprise a stent disposed between the inner member and the outer sheath in a first configuration. The stent may be configured to transition between the first configuration and a second configuration when deployed from between the inner member and outer sheath. In the second configuration, the stent may comprise at least four retention members, including second and third retention members each having at least one shoulder separated by a cylindrical saddle region. The at least four retention members may include first and fourth retention members disposed respectively on a side of the second and third retention members furthest from the cylindrical saddle region. The first retention member, second retention member, third retention member, fourth retention member, or any combination thereof may be configured to atraumatically interface with tissue. The system may include a handle configured to fix and unfix (e.g., lock and unlock) the inner member and the outer sheath with respect to each other. The handle may be configured to independently deploy each of the at least four retention members of the stent at respective target locations.

In the above and other aspects, the first and fourth retention members may be configured to atraumatically interface with tissue. The first and fourth retention members may be configured to atraumatically interface with tissue about a radially outwardmost circumference of the respective retention member. The stent may be formed from at least one braided or woven filament. The at least one braided or woven filament may comprise a different pitch density, angle, or pattern at the first retention member, the second retention member, the cylindrical saddle region, the third retention member, or the fourth retention member, or any combination thereof, in the second configuration. The stent may comprise at least a partial cover. The first retention member, the second retention member, the first retention member, or the fourth retention member, or any combination thereof, may comprise an asymmetrical cross section in an orthogonal plane to a longitudinal axis extending through a lumen of the stent.

### BRIEF DESCRIPTION OF THE DRAWINGS

Non-limiting examples of the present disclosure are described with reference to the accompanying figures, which are schematic and not intended to be drawn to scale. In the figures, each identical or nearly identical component illustrated is typically represented by a single numeral. For purposes of clarity, not every component is labeled in every figure, nor is every component in each embodiment of the disclosure shown where illustration is not necessary to allow those of skill in the art to understand the disclosure. In the figures:

FIG. 1A illustrates an example of a stent deployed in a body lumen according to embodiments of the present disclosure.

FIG. 1B illustrates dimensional details of the stent of FIG. IA.

FIG. 2 illustrates an example of a stent with a partial covering according to embodiments of the present disclosure.

FIG. 3A illustrates an example of a stent with a varying weave pitch according to embodiments of the present disclosure.

FIG. 3B illustrates an example of an aperture of a weave according to embodiments of the present disclosure.

FIGS. 4A-4B illustrate various dimensional aspects of cross sections of retention members in examples according to embodiments of the present disclosure.

FIGS. 5A-5M illustrate cross-sectional profiles of various examples of retention members according to embodiments of the present disclosure.

FIGS. 6A-6K illustrate side views of various examples of retention members according to embodiments of the present disclosure.

FIGS. 7A-7B illustrate examples of a mandrel and clamp as may be useful for manufacturing stents according to embodiments of the present disclosure.

FIG. 8 illustrates a delivery system according to embodiments of the present disclosure.

FIGS. 9A-9E illustrate an example of a staged deployment of a stent according to embodiments of methods of the present disclosure.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical devices (e.g., stents, etc.) and systems for drainage of fluids through lumens such as the esophagus, or the like, it should be appreciated that such medical devices may be used in a variety of medical procedures (e.g., external biliary drain conversion, enteroenterostomy, gastroduodenostomy and gastroileostomy, etc.) to establish and/or maintain a temporary or permanent open flow or drainage passage in, from, or between a variety of body organs, lumens, ducts, vessels, fistulas, cysts and spaces (e.g., the esophagus, dermis, stomach, duodenum, jejunum, small intestine, gallbladder, kidneys, pancreas, biliary/pancreatic trees, bladder, ureter, abscesses, walled-off pancreatic necrosis (WOPN), bile ducts, etc.). The devices may be inserted via different access points and approaches, e.g., percutaneously, endoscopically, laparoscopically or some combination thereof. The medical devices disclosed herein are self-expanding, but in other embodiments the medical device may be expandable by other means, including, e.g., a balloon catheter. Moreover, such medical devices are not limited to drainage, but may facilitate access to organs, vessels or body lumens for other purposes, such as creating a path to divert or bypass fluids or solids from one location to another, removing obstructions and/or delivering therapy, including non-invasive or minimally invasive manipulation of the tissue within the organ and/or the introduction of pharmacological agents via the open flow passage.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

In embodiments, the present disclosure relates to a medical device (e.g., self-expanding metal stent and/or a duodenal exclusion device, etc.) configured to extend within a body lumen and assist with maintaining a temporary or permanent open path for fluid flow therethrough. The medical device may include at least four retention members configured to appose tissue and thereby resist migration of the medical device.

One or both of second and third retention members along opposing longitudinal sides of the stent, as described herein, such as flanges, may be double-walled and include one or more substantially perpendicular or non-perpendicular surfaces of an axially inward and axially outward wall of the flange, the walls oriented with respect to a cylindrical saddle region extending along a longitudinal axis between the flanges. Such a configuration of non-perpendicular surfaces may reduce migration of the medical device with respect to the tissue(s) apposed or engaged by the flanges, when compared, for example, to a corresponding retention member with only perpendicular surfaces. Additionally, or alternatively, retention members, as double-walled flanges with one or more non-perpendicular surfaces, may be configured to provide more control over the resistance of the device being pulled out of its intended placement once deployed, e.g., resulting in higher pull-out forces as compared to a corresponding retention member with perpendicular surfaces. In various embodiments, one or more non-perpendicular surfaces may also be configured to interact less traumatically with at least one tissue wall of the first and second body lumens (e.g., configuring an inward wall with a point of tissue contact having less surface area), as compared, for example, to a perpendicular surface or to another surface with at least one tissue-engaging element, such as a prong, barb, hook, or other like design.

One or both of first and fourth retention members along opposing longitudinal sides of the stent, as described herein, such as flared regions, may additionally appose tissue(s) and thereby reduce a risk of migration of a stent. In various embodiments, at least first and fourth retention members may be configured to atraumatically interface with apposed or engaged tissue. In various embodiments, first and/or fourth retention members may be configured to reduce a pressure between tissue and respectively apposed or engaged second and/or third retention members. Without wishing to be bound by any particular theory, it is thus believed that first and fourth retention members such as those described herein may reduce a risk of injury of a stent to apposed or engaged tissue while reducing a risk of stent migration. It will be appreciated that varying degrees of engagement of one or more of the first, second, third, and/or fourth retention members may be customized by adjusting a geometry, radial strength, or the like of either the respective or at least one alternative retention member.

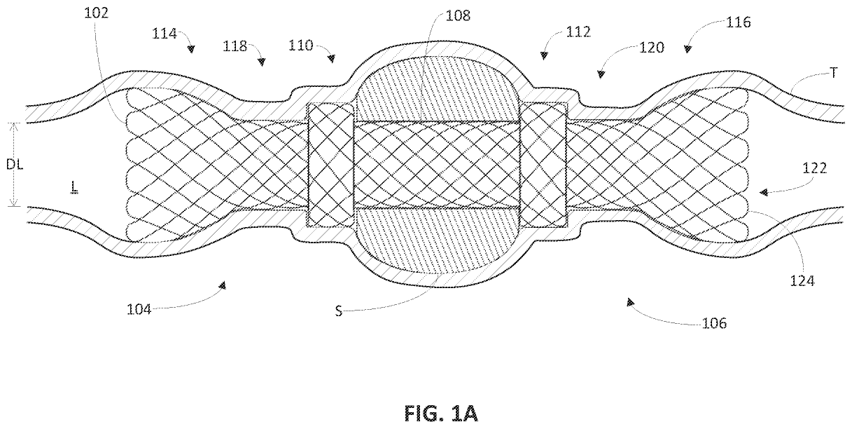

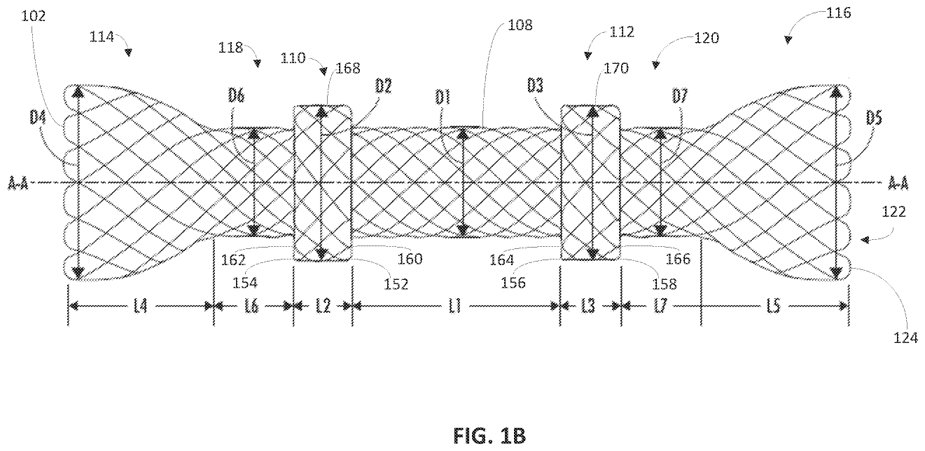

Turning to the drawings, FIG. 1A illustrates a stent 102 according to various examples described herein. Stent 102 is positioned within tissue “T” defining lumen “L” with a natural lumen diameter “DL.” It will be understood that, in alternative examples, stents according to the present disclosure may be positioned across multiple tissues and/or lumens, such as in an end-to-end anastomosis, and end-to-side anastomosis, or a side-to-side anastomosis. In various examples, stent 102 may be positioned across target tissue and/or treatment area, such as stricture “S.” Embodiments are not limited in this context.

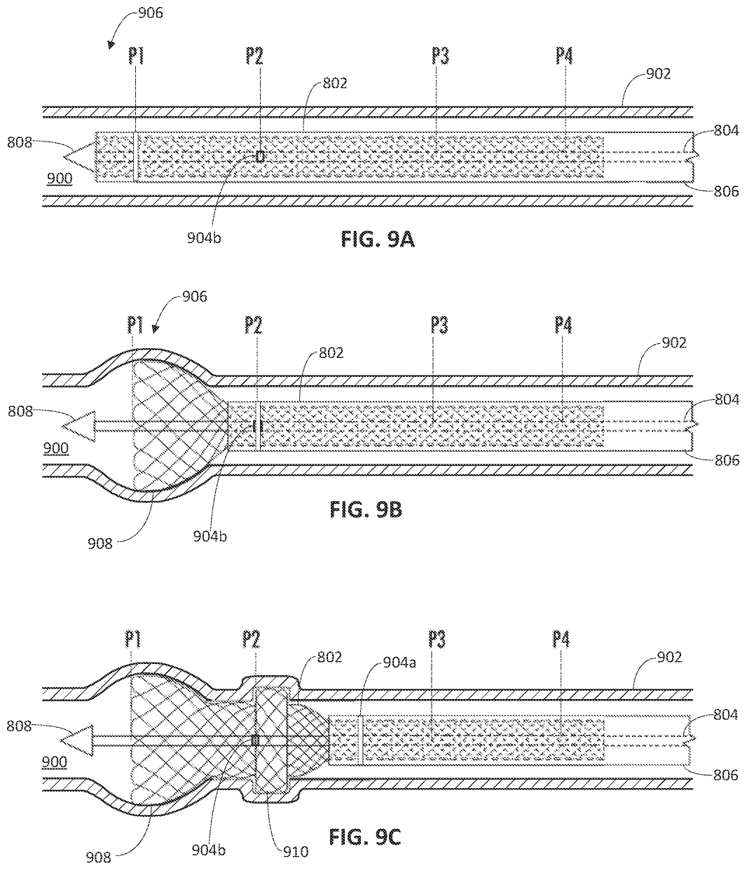

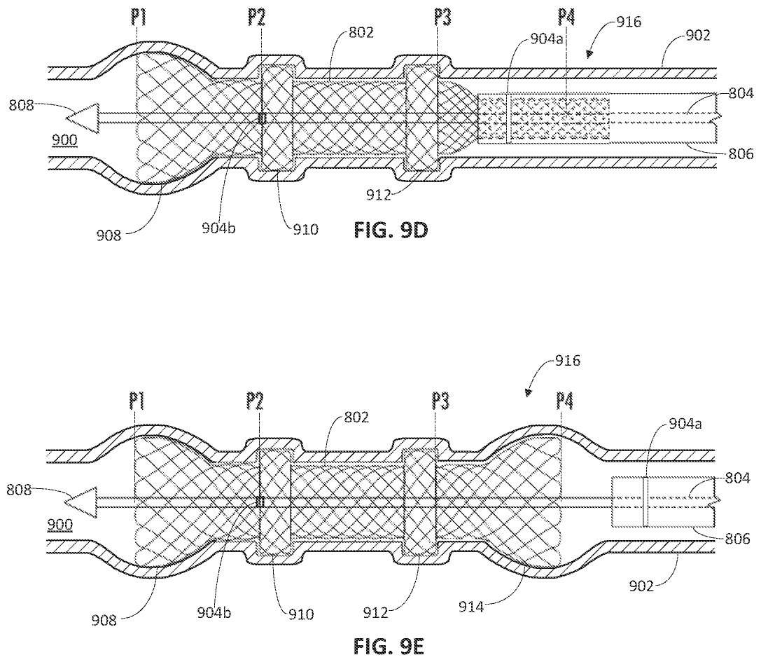

In some embodiments, stents according to the present disclosure may comprise at least one solid surface, for example, a polymeric or silicone surface (not shown). In some embodiments, stents such as stent 102 may be formed entirely or partially of at least one filament braid, such as a polymeric or metallic (e.g., Nitinol) wire. In various examples, stents may comprise a shape memory material. For example, stent 102 is formed of at least one wire 124 which may be pre-formed in a deployed configuration (e.g., as illustrated in FIGS. 1A-B) and subsequently constrained or otherwise loaded into a delivery system, as illustrated in and discussed with respect to FIG. 8 and/or FIGS. 9A-E.

Stent 102 may include a first end 104, a second end 106, and a middle region 108 (e.g., saddle region, cylindrical saddle region, or the like) extending therebetween. In various examples, stent 102 may include at least four retention members configured to appose tissue such as tissue T.

In various embodiments, one or both of first end 104 and second end 106 may include multiple retention members. For example, first end 104 may include first retention member 114 and second retention member 110. Second end 106 may include third retention member 112 and fourth retention member 116.

Various embodiments may include at least two tissue-apposing shoulders, flanges, or the like as retention members. For example, retention members 110, 112 of stent 102 may comprise tissue-apposing shoulders. As illustrated in FIG. 1B, retention member 110 may include faces 160, 162 which are each transverse to axis A-A. Face 160 may be an axially inner face and face 162 may be an axially outer face (i.e., along axis A-A). In some embodiments, retention member 110 may continue from faces 160, 162 respectively into corners 152, 154 (i.e., forming shoulders), which may join together directly (not shown) or by an intervening segment of stent body such as cylindrical portion 168. It will be understood that intervening segments of stent body within retention members of the present disclosure may include one or more straight and/or curved portions. In various embodiments, intervening segments of stent body such as cylindrical portion 168 may form radially outward surfaces configured to interface with tissue. For example, retention members as described herein may be configured to interface with tissue about a radially outwardmost circumference. Embodiments are not limited in this context.

Similarly, retention member 112 may include faces 164, 166 which continue respectively into corners 156, 158. Face 164 may be an axially inner face and face 166 may be an axially outer face (i.e., along axis A-A). Corners 156, 158 may join directly together (not shown) or by an intervening segment such as cylindrical portion 170.

In some embodiments, multiple retention members may be directly adjacent to one another (i.e., without space along the stent therebetween). In other embodiments, such as with stent 102, retention members may be separated by intervening extents of stent body which may be cylindrical, ramped, undulating, or otherwise shaped. For example, saddle region 118 extends between first retention member 114 and second retention member 110, and saddle region 120 extends between third retention member 112 and fourth retention member 116.

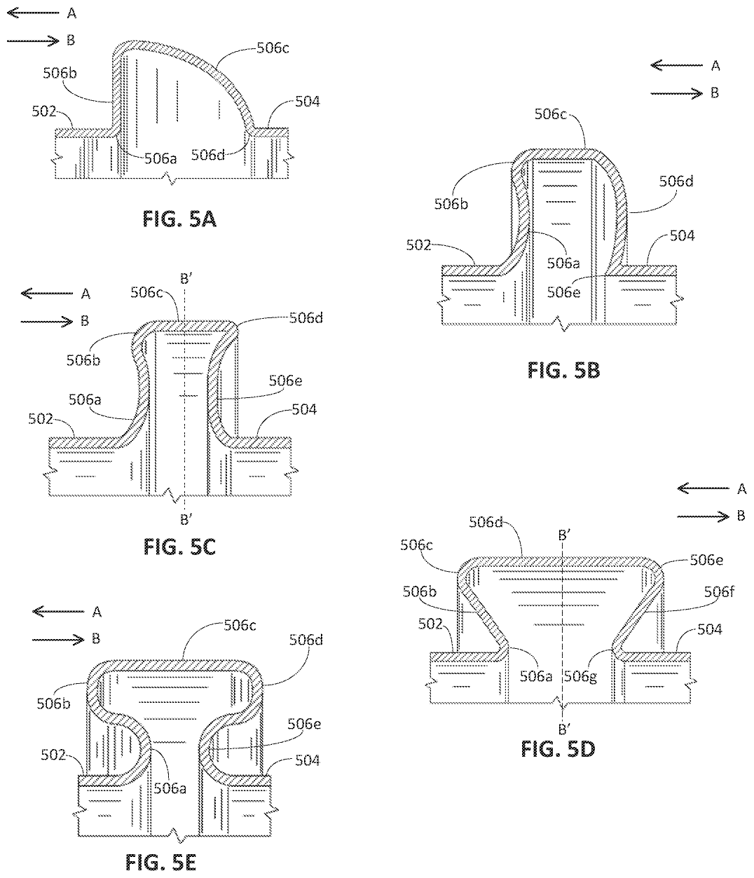

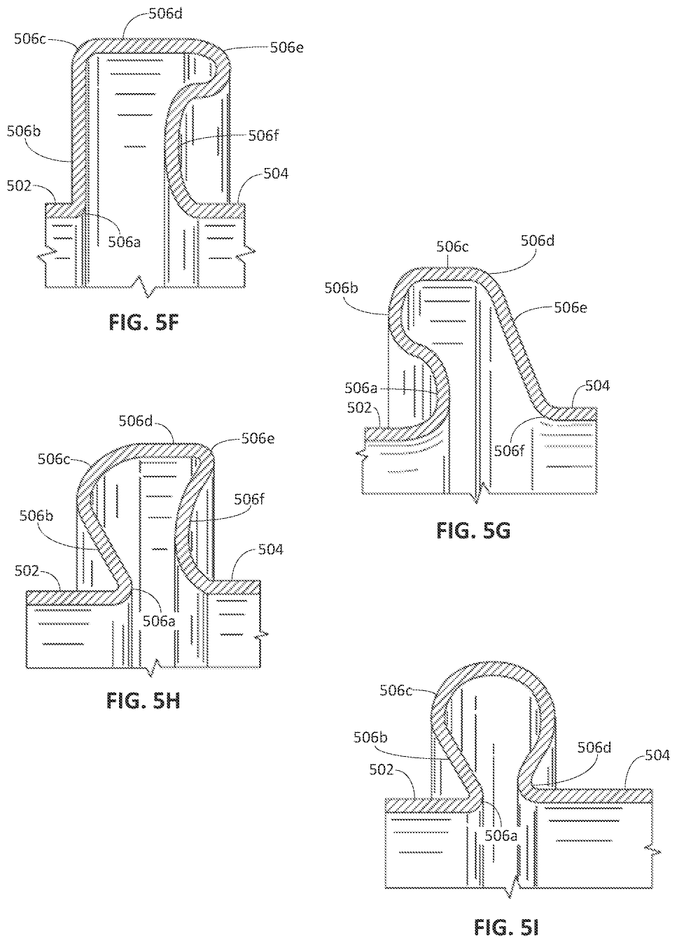

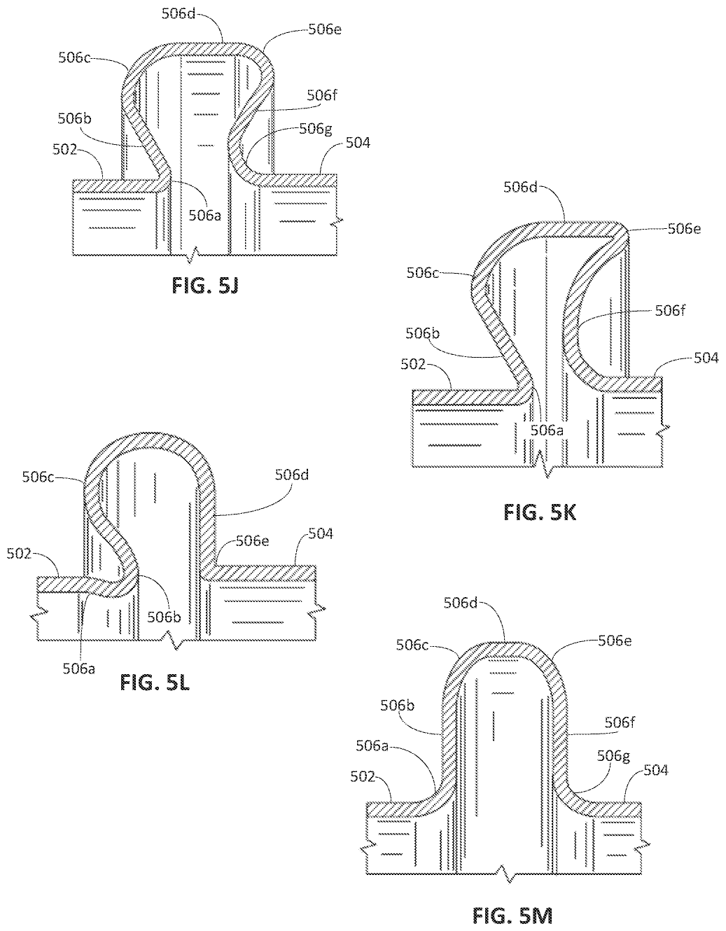

Retention members according to the present disclosure may include various geometries, dimensions, angles, radii of curvature, or the like along portions thereof in order to form a tissue-apposing or engaging interface with a desired conformation and/or lack thereof to a tissue surface, radial strength, pull-out force, or the like. For example, as described with respect to FIGS. 5A-M, faces of retention members may be set at different angles with respect to a surface of a saddle region, such as middle region 108, saddle region 118, or saddle region 120. It will be recognized that retention members of the present disclosure may accordingly be designed to interface more or less traumatically with apposed or engaged tissue.

In various embodiments, one or more of first, second, third, or fourth retention members may comprise different shapes, dimensions, or the like from each other. For example, second and third retention members 110, 112 of stent 102 comprise tissue-apposing shoulders and first and fourth retention members 114, 116 comprise flared regions, which may each be configured to interface with tissue along a partial or full longitudinal length thereof, for example, about a radially outward surface thereof. It will be appreciated that various shapes, dimensions, materials (e.g., polymeric and/or metallic filaments), material arrangements (e.g., weave pitch, braid pattern, number of filaments, or the like) may present alternative mechanical characteristics such as flexibility, radial strength/force, or the like. Accordingly, any or each of retention members 110, 112, 114, 116 may be configured so as to provide customized mechanical characteristics to stent 102. Similarly, each of middle region 108, saddle region 118, or saddle region 120 may comprise one or more similarities or differences to each other.

According to some embodiments, stent 102 defines a lumen 122 extending longitudinally therethrough (i.e., extending continuously through first end 104, middle region 108, and second end 106 along axis ‘A-A’, as shown in FIG. 1B). Accordingly, stent 102 may facilitate fluid flow through lumen L of tissue T.

FIG. 1B illustrates a side view of stent 102 with various aspects and dimensions which may be independently or jointly customized. For example, middle region 108 has a longitudinal length “L1” (i.e., as measured along longitudinal axis A-A) and a maximum diameter “D1.” Second retention member 110 comprises a longitudinal length “L2” and a maximum diameter “D2,” and third retention member 112 comprises a longitudinal length “L3” and a maximum diameter “D3.” First retention member 114 comprises a longitudinal length “L4” and a maximum diameter “D4,” and fourth retention member 116 comprises a longitudinal length “L5” and a maximum diameter “D5.” Saddle region 118 comprises a longitudinal length “L6” and a maximum diameter “D6,” and saddle region 120 comprises a longitudinal length “L7” and a maximum diameter “D7.”

In various embodiments, L2 and L4 may be substantially the same, L3 and L5 may be substantially the same, or both. In some examples, L3 may be greater than L2, for example, 5%, 10%, 15%, 20%, 25%, 30%, 35%, 40%, 45%, 50%, 55%, 60%, 65%, 70%, 75%, 80%, 85%, 90%, 95%, or 100% greater, although alternative dimensions are additionally contemplated. Likewise, L5 may be independently greater than L4. In other examples, L3 and/or L5 may be smaller than one or both of L2 and/or L4, such as 5%, 10%, 15%, 20%, 25%, 30%, 35%, 40%, 45%, 50%, 55%, 60%, 65%, 70%, 75%, 80%, 85%, 90%, or 95% smaller. Embodiments are not limited in this context.

In some embodiments, L6 may be at least as great or greater than one or both of L2 or L4, L7 may be at least as great or greater than one or both of L3 or L5, or both. In various embodiments, L6 and L7 may be substantially the same. L6 and/or L7 may be at least as great as or substantially the same as L1. In other embodiments, L6 and/or L7 may be 20%, 25%, 30%, 40%, 45%, 50%, 55%, 60%, 65%, 70%, 75%, 80%, 85%, 90%, 95%, 105%, 110%, 115%, 120%, 125%, 130%, 135%, 140%, 145%, 150%, or another percentage of L1. Embodiments are not limited in this context.

In various examples according to the present disclosure, one or more of diameters D1, D2, D3, D4, D5, D6, or D7 may be at least as large in magnitude as DL. Thus, respective aspects of stent 102 may be configured to interface with tissue T and/or stricture S so as to lodge stent 102 within lumen L. In some embodiments, at least one or more of D2, D3, D4, or D5 may be larger that DL so as to apply a radially outward pressure to tissue T. In various examples, such as that illustrated in FIG. 1A, one or more of retention members 110, 112, 114, or 116 may accordingly distend or securely engage against a correspondingly engaged portion of tissue T. It will be recognized that such application of radial pressure on tissue T by aspects of stent 102 with an outer perimeter extending beyond the natural resting position of tissue T (i.e., a maximum diameter larger than DL) may further reduce a tendency of stent 102 to migrate within lumen L. In some embodiments, for example, a corner or edge of one or more of retention members 110, 112, 114, or 116 may resist migration based on a friction or interference fit against tissue T.

It will be recognized that adjustment of one or more of diameters D1, D2, D3, D4, D5, D6, or D7 and/or lengths L1, L2, L3, L4, L5, L6, or L7 may affect the interaction of stent 102 with tissue T variously and/or variably along its length. For example, if D4 is significantly larger than D2, the first retention member 114 may somewhat or substantially lift a surface of tissue T away from the surface of stent 102 and thereby reduce a pressure of part or all of second retention member 110 against tissue T. Accordingly, one or both of corners 152, 154 may less traumatically engage tissue T. The effect of a relatively large diameter D4 with respect to diameter D2 may be increased by reducing length L6, as tissue T may be somewhat more stretched across the saddle region 118. However, increasing length L6 may allow tissue T to more closely conform to saddle region 118, focusing distensions or secure engagement of tissue T at retention members 110, 114 and thereby increasing a friction between retention members 110, 114 to tissue T and/or a trauma of retention members 110, 114 to tissue T along part or all of their respective lengths. Embodiments are not limited in this context.

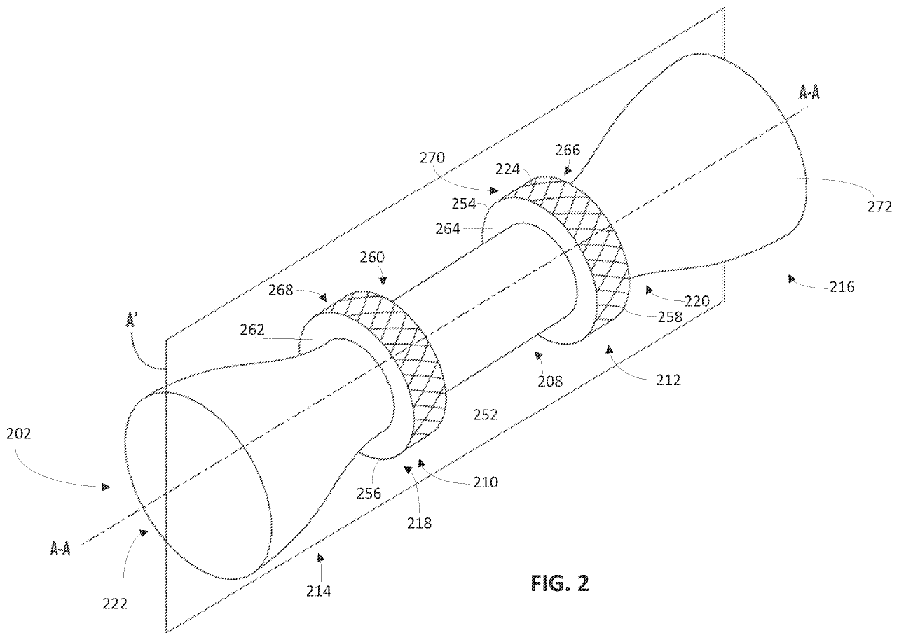

Various stents according to the present disclosure may comprise full or partial coverings, coatings, or the like. For example, as illustrated in FIG. 2, stent 202 may comprise one or more similarities as stent 102 as discussed above but further include a partial covering. In particular, retention members 210, 212, 214, and 216 may respectively correspond to retention members 110, 112, 114, and 116 and comprise similar dimensions, material properties, and/or mechanical characteristics. For example, faces 260, 262, 264, and 266 respectively correspond to faces 160, 162, 164, and 166, and corners 252, 254, 256, and 258 respectively correspond to corners 152, 154, 156, and 158. Similarly, middle region 208 and saddle regions 218, 220 may respectively comprise one or more similarities with middle region 108 and saddle regions 118, 120, axis A-A may extend longitudinally through stent 202. Stent 202 may be formed of filament braid 224 and define a lumen 222 extending therethrough.

However, stent 202 comprises a partial covering 272 which extends along retention member 114, saddle region 118, and face 254, as well as face 260, middle region 208, face 264, face 266, saddle region 120, and retention member 216. Accordingly, cylindrical regions 268, 270, as well as corners 252, 254, 256, and 258 are uncovered, with filament braid 224 exposed to directly interface with apposed or engaged tissue.

Where present, covering 272 may extend between or across at least one aperture of a weave pattern of filament braid 224, thereby preventing tissue ingrowth and/or fluid leakage therethrough. In other examples, covering 272 may extend across cylindrical region 268 and/or 270 additionally or alternatively to the covered regions illustrated in FIG. 2. Without wishing to be bound by any particular theory, it is believed that uncovered regions of stents formed from filament braid(s) may enable tissue ingrowth which reduces a risk of stent migration. Accordingly, various examples may include one or more of uncovered retention member 214, 216, face 260, 262, 264, 266, or middle region 208 in order to increase a pull-out force over a corresponding covered stent and/or reduce a risk of migration of the stent (e.g., as a result of a patient's natural movement).

In some embodiments according to the present disclosure, one or more sections of stent 202 may be uncovered along tissue-apposing portions of the stent. For example, one or more of retention members 210, 212, 214, 216 may be uncovered along at least a radially outwardmost surface (e.g., cylindrical regions 268, 270 of respective retention members 210, 212) configured to interface with tissue T. It will be appreciated that various patterns, extents, and/or materials of covering may be applied to one or more portions of stent 202 in order to customize an interaction of stent 202 with tissue T, for example, to achieve a desired pull-out force and/or retentive strength of the stent. Embodiments are not limited in this context.

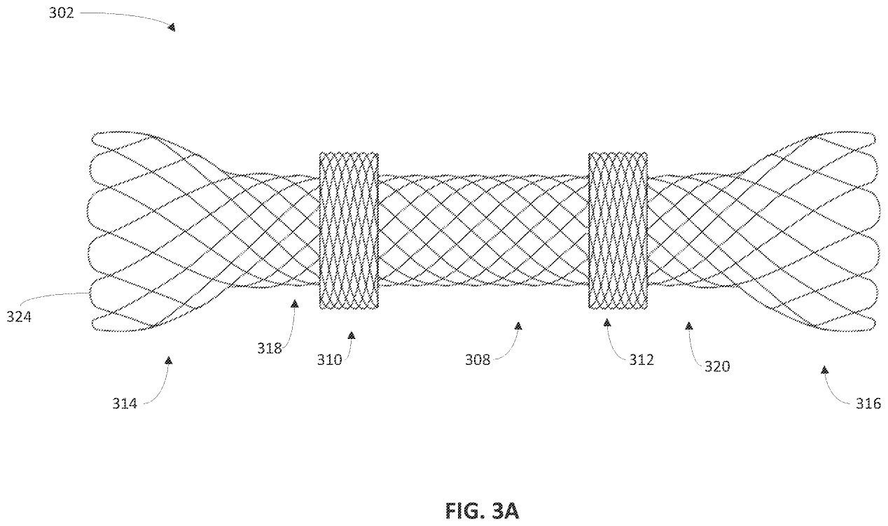

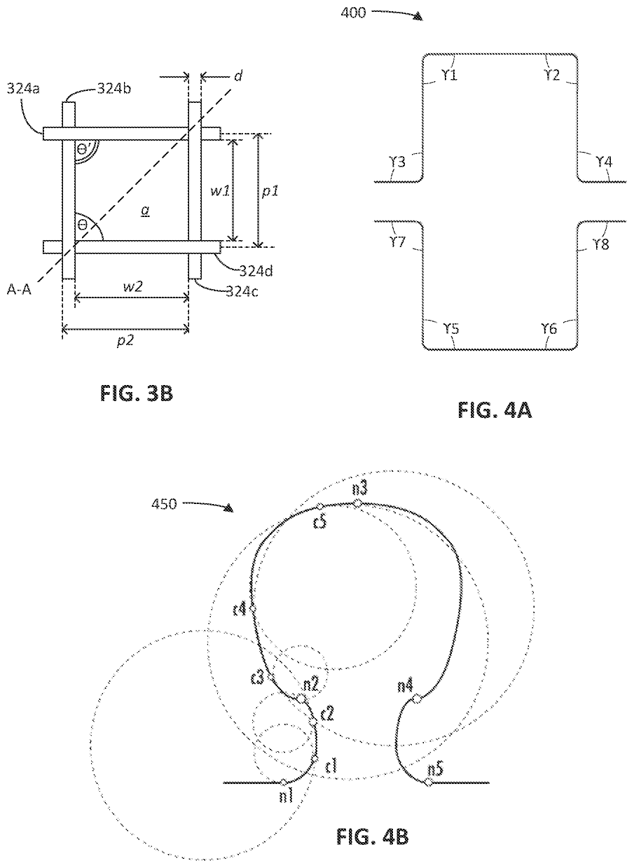

FIG. 3A illustrates an additional example of a stent 302 formed of at least one filament braid 324 with varying pitch. Various examples of stents according to the present disclosure may include two or more portions having differing pitches of filament braid, differing weave pattern, differing number of filaments woven together, and/or filament material, which may each comprise a respectively different radial strength, flexibility, susceptibility or resistance to tissue ingrowth, coefficient of friction, or other property known to affect an implant interface with tissue, mechanical behavior of the implant, or both.

As described herein, pitch density of a filament braid refers to the closeness of the filaments forming the braid. For example, a braid may have a more or less dense pitch based on a spacing between filaments, an angle between two or more filaments of the braid, or other arrangements of filament(s) of a braid. For example, as illustrated in FIG. 3B, at least one filament braid 324 (i.e., filament), comprising at least segments 324a, 324b, 324c, and 324d, of diameter “d” is braided (or alternatively woven) to define an aperture “a” defined by sides of widths “w1” and “w2” and set at angles “Θ” and “Θ′” with respect to each other. Pitch density may be defined based on the lesser of “p1” and “p2,” each of which may respectively be defined as the sum of a maximum distance between filament segments defining opposite sides of aperture a with the diameter d of filament braid 324. Accordingly, pitch density may be affected by changing one or more of w1, w2, Θ, or Θ′. While not illustrated for the sake of simplicity in the drawings, it is further contemplated that various filaments of differing diameters may additionally be woven into a stent according to the present disclosure, and that such differing diameters may additionally or alternatively affect pitch density.

Weave pitch angle, as described herein, may be defined by the extent of the interior opposing angles of the individual apertures formed by the filament(s). For example, weave pitch angle may be defined by the greater of Θ, or Θ′, or weave pitch angle may be defined by the angles of an aperture which most directly oppose a longitudinal axis of a stent, such as axis A-A of stent 202. For example, aperture a is set at an offset angle to axis A-A in the illustration of FIG. 3B. Θ′ opposes axis A-A more (e.g., more directly) than Θ. Accordingly, weave pitch angle for aperture a may be defined by or as Θ′. While not illustrated for the sake of simplicity, it is presently contemplated that various embodiments may comprise one or multiple weave pitch angles along their extent. For example, at least one retention member and/or saddle region of a stent may comprise an alternative weave pitch pattern than that of other parts of the stent. Varying weave pitch angles may, for example, provide alternative retentive strengths and/or flexibilities along the length of a stent.

Weave pattern, as described herein, refers to how segments of filament(s) are woven together and/or oriented with respect to each other. For example, a weave pattern may comprise a particular over:under pattern, in which a segment may pass over a first number of crossed segments and then under a second number of crossed segments. For example, FIG. 3B may comprise a 1:1 weave pattern, in which segment 324a passes over one segment (segment 324b) before passing under one segment (segment 324c). Likewise, segment 324d passes under one segment (segment 324b) before passing over one segment (segment 324c). Various additional or alternative weave patterns are presently contemplated, for example, 1:2, 1:3, 1:4, 1:5, 1:6, 1:7, 1:8, 1:9, 1:10, 2:2, 2:3, 2:4, 2:5, 2:6, 2:7, 2:8, 2:9, 2:10, 3:3, 3:4, 3:5, 3:6, 3:7, 3:8, 3:9, 3:10, 4:4, 4:5, 5:5, 5:6, 5:7, 5:8, 5:9, 5:10, 6:6, 6:7, 6:8, 6:9, 6:10, 7:7, 7:8, 7:9, 7:10, 8:8, 8:9, 8:10, 9:9, 9:10, and 10:10 weave patterns.

Some embodiments may comprise multiple weave patterns. For example, one or more of a retention member or saddle region may comprise a different weave pattern than another portion of a stent. In another example, various filaments or segments of a filament may comprise alternative weave patterns. For example, segment 324a may comprise a 1:1 weave pattern, as illustrated in FIG. 3B, but filament 324d may pass over an additional segment (not illustrated for the sake of simplicity in the drawings) immediately after segment 324c and thereby comprise a 1:2 weave pattern. Embodiments are not limited in this context. Varying weave patterns may, for example, provide alternative retentive strengths and/or flexibilities along the length of a stent.

Stent 302 comprises retention members 310, 312 with a higher density filament pitch (i.e., closer weave) than a filament pitch of middle region 308, saddle region 318, and/or saddle region 320. Without wishing to be bound by any particular theory, it is believed that a portion of a stent body with a higher density filament pitch may contribute to a higher radial strength (i.e., ability to withstand pressure applied radially inward without collapsing) than a corresponding portion of a stent body with a lower density filament pitch. For example, retention members 310, 312 may comprise higher radial strengths than middle region 308.

Stent 302 additionally comprises retention members 314, 316 with a lower density pitch (i.e., looser weave) than the filament pitch of middle region 308, saddle region 318, and/or saddle region 320. Accordingly, retention members 314, 316 may comprise a lower radial strength than one or more of retention member 310, retention member 312, middle region 308, saddle region 318, and/or saddle region 320. In some embodiments, stent portions such as retention members 314, 316 may be configured to atraumatically interface with tissue based on a lower pitch density. For example, retention members 314, 316 may comprise combined benefits of resistance to migration based on their relatively wide diameters and increased compliance with and reduced trauma to apposed or engaged tissue based on their relatively low pitch density. Embodiments are not limited in this context.

Various embodiments include retention members with one or more geometric features, including stent wall angle, shape, radius of curvature, or the like, which may contribute to a pull-out strength of the respective retention member. For example, FIG. 4A illustrates an example of a profile of a retention member 400 such as any of retention members 110, 112, 210, 212, 310, 312, for example, as viewed along plane A′, parallel to axis A-A, as illustrated in FIG. 2. As illustrated in FIG. 4A, retention member 400 may include one or more of angles “Υ1,” “Υ2”, “Υ3,” “Υ4,” “Υ5,” “Υ6,” “Υ7,” or “Υ8” (collectively referred to herein as Υ1-8, which will be understood to refer to any or each of Υ1, Υ2, Υ3, Υ4, Υ5, Υ6, Υ7, or Υ8) defined by respectively adjacent segments of stent wall forming retention member 400. While each of Υ1-8 is illustrated in FIG. 4A as approximately may be less than or greater than 90 degrees.

In various embodiments, a retentive strength and/or pull-out force of retention member 400 may be higher in accordance with an acuteness of one or more of Υ1-8. For example, a dislodgement of retention member 400 from within tissue may require a longitudinal stretching and/or deformation of retention member 400 (e.g., so as to decrease a maximum diameter thereof, such as D2 or D3 as discussed above). A more acute angle Υ1-8 may correspond with its defining adjacent segments of stent wall oriented so as to generate an opposing force to such a lateral deformation. For example, it will be understood that a directional force “F 1” applied to retention member 400 (e.g., as may be applied by pulling the right side of the retention member 400 further right) may result in an opposing directional force “F2” applied by the segment of wall extending between Υ2 and Υ4 if one or both of Υ2 and Υ4 are less than 90 degrees. The opposing force F2 may thus resist the deformation and resulting dislodgement of retention member 400.

Υ1-8 may each comprise the same angle, or one or more of Υ1-8 may comprise at least one different angle from each of the others. While retention members herein are illustrated as being circumferentially symmetric (e.g., about axis A-A) for the sake of simplicity, it is further presently contemplated that one or more stents may include retention members comprising asymmetrical cross sections orthogonal to a longitudinal axis extending therethrough (e.g., axis A-A). For example, Υ1 and Υ5 may comprise different angles from each other, Υ1 and Υ2 may comprise different angles from each other, Υ3 and Υ7 may comprise different angles from each other, or any other combination of Υ1-8 may comprise different angles from each other.

Accordingly, a pull-out force of a retention member may be customized based on its interior angles. Additionally, or alternatively, it will be recognized that more acute angles Υ1-8 may reduce a conformation of retention member 400 to apposed or engaged tissue, which may, in some embodiments, allow the retention member 400 to more effectively latch onto and/or into apposed or engaged tissue.

Similarly, it will be recognized that more obtuse angles Υ1-8 (i.e., over 90 degrees) may reduce a respective retention force of retention member 400 and/or increase a conformation of retention member 400 to apposed or engaged tissue. For example, one or more of Υ1-8 may be designed to be more obtuse than in alternative configurations so as to reduce a trauma of the respective portion of retention member 400 with apposed or engaged tissue.

Intervening sections of stent wall between respective Υ1-8 may extend directly between the same as straight segments (as illustrated in FIG. 4A) or as non-straight segments (as illustrated in FIG. 4B). In some embodiments, intervening segments may include one or curves, for example, defining any number of additional inflection points, angles Y9-x with x representing any positive integer, or the like.

For example, FIG. 4B illustrates an example of a profile of one side of retention member 450 such as any of retention members 110, 112, 210, 212, 310, 312, taken along one half of plane A′ as intersected by axis A-A. Retention member 450 includes various inflection points n1, n2, n3, n4, n5, which are points along retention member 450 at which a direction of curvature changes (i.e., a stent shape transitions between any two of a concave, a convex, and a straight portion). Retention member 450 may include any positive integer y of inflection points. While not illustrated in FIG. 4B for the sake of simplicity, angles Υ1, 2, 3 . . . x may be defined in retention member 450 between tangent lines extending through inflection points nearest to each other.

Υ1, 2, 3 . . . x and/or segments extending between any of n1, 2, 3 . . . y may comprise one or more smooth curves, each of which may comprise at least one internal radius of curvature. In some embodiments, various curves of stent wall may be approximated based on approximate radii of curvature which they define. For example, intersections between curves are indicated along the left side retention member 450 as c1, c2, c3, c4, and c5, with each of nl-y additionally indicating intersections of adjacent curved portions, although any positive integer z of curves may be defined within retention member 450. Dotted circles superimposed over variously defined curves of retention member 450 (e.g., between n1 and c1, between c1 and c2, between c2 and n2, between n2 and c3, between c3 and c4, between c4 and c5, and between c5 and n3) approximate the respective curves and each comprise a respective radius (not shown for the sake of simplicity) defining the radius of curvature of the respective curves. Any or each of the curves of retention member 450 may comprise the same or different radius of curvature.

As described with respect to retention member 400 above, retention member 450 may be symmetric or asymmetric. For example, each side of retention member 450 may comprise similar and/or different inflection points, curves, radii of curvature, or the like.

FIGS. 5A-5M illustrate additional examples of profiles of retention members positioned between saddle regions of a stent, such as any of retention members 110, 112, 210, 212, 310, and 312 taken along one half of plane A′ as intersected by axis A-A. Each of FIGS. 5A-5M includes two saddle regions 502, 504, each of which may respectively correspond with saddle region 118 and middle region 108, 208, 308 or middle region 108, 208, 308 and saddle region 120. Each of FIGS. 5A-5M further includes various portions 506 defined between stent wall inflection points (such as n1-5, not shown in FIGS. 5A-5M for the sake of simplicity). For the sake of simplicity, subsequently described portions 506 of FIGS. 5A-5M will be referred to as 506a, 506b, 506c, 506d, and the like. However, it will be appreciated that any number and/or configuration of portions 506 may be included in retention member configurations according to the present disclosure.

With respect to FIG. 5A, the saddle region 502 may extend into a portion 506b through a portion 506a at approximately a right angle. In this example, saddle region 502 and portion 506b form a concave curve with a small radius of curvature (e.g., less than 0.2 mm) that may more or less be a fold (i.e., forming an interior angle at a single point corresponding to 506a). Portion 506b may comprise a straight edge extending transverse (e.g., substantially perpendicular) to a longitudinal axis extending through the stent (such as axis A-A). Portion 506b may thus form a face 154, 160, 164, 166, 254, 260, 264, 266 as described above. Portion 506b may have a length between 10% and 300% the diameter of the stent, in various examples. Portion 506b may then extend into portion 506c. In FIG. 5A, portion 506c bends convexly over the stent lumen to decrease in diameter to approximately the diameter of saddle region 504, at which it is connected to saddle region 504 via portion 506d. Portion 506d comprises a concave curve with a tight radius of curvature as discussed as with respect to portion 506a.

Accordingly, each side of a retention member with a configuration as illustrated in FIG. 5A may be seen to have an approximately half “D” shape, with portion 506c functioning as one or both of one of faces 154, 160, 164, 166, 254, 260, 264, 266 and one of cylindrical regions 260, 270 as described above. It will be appreciated that the configuration of FIG. 5A may thus present one or more benefits over alternative configurations, such as a greater resistance of the retention member to migrate in a direction of arrow “A” as opposed to in a direction of arrow “B.” The differential in tendency to migrate may be based on one or both of a non-conformance of combinations of saddle region 502, portion 506a and/or portion 506b to a natural shape of apposed or engaged tissue (not shown) or a conformance of portion 506c to the natural shape or tendency of apposed or engaged tissue to return to its natural shape. For example, conformance of portion 506c to apposed or engaged tissue may be enabled based on its gradual change in diameter along its length.

FIG. 5B illustrates an additional example of a retention member with two sides similarly configured to resist the same directional forces as each other. In FIG. 5B, saddle region 502 extends into portion 506a, which continues concavely radially outward with a relatively wide radius of curvature to form a wall such as one of faces 154, 160, 164, 166, 254, 260, 264, or 266 as described above. For example, portion 506a may have a radius of curvature which is 25-200% of a diameter of saddle region 502, saddle region 504, or both. Portion 506a then extends into portion 506b, which forms a convex curve into portion 506c, which may correspond to one of cylindrical regions 268, 270 as described above.

In various embodiments, portion 506a may extend concavely to an extent that it bends back over itself and/or saddle region 502, to an extent that portion 506b extends over part of portion 506a, saddle region 504, or both. Portion 506a, portion 506b, or the combination thereof may thus not conform to a tissue surface apposed or engaged along at least portion 506c. As discussed above, the changes in direction and overlapping portions of stent wall between one or more of saddle region 502, portion 506a, and portion 506b may resist a pull-out force in the direction of arrow A via a resultant force in portion 506b in the direction of arrow B.

Portion 506c may continue into portion 506d, which may convexly curve to decrease in diameter. Portion 506d may form one of faces 154, 160, 164, 166, 254, 260, 264, or 266 as described above. Portion 506d may extend convexly to an extent that it bends back under itself and/or portion 506c. Portion 506d may thus extend into saddle region 504 through portion 506e at an acute interior angle.

It will be recognized that the similar profiles of portions 506a, 506d may enable each of them to similarly resist a force in the same direction. For example, if the retention member is subjected to a force in direction A, each of portions 506a, 506d may facilitate a resistive opposing force in direction B. Accordingly, a configuration such as illustrated in FIG. 5B may present improvements over alternative retention members in environments in which resistance to migration, dislodgement, or both in a single direction is preferred. Embodiments are not limited in this context.

The example of FIG. 5C may provide asymmetric resistance to migration and/or dislodgment in different directions. In FIG. 5C, one or more of saddle region 502, portion 506a, portion 506b, and portion 506c may be oriented as discussed above with respect to FIG. 5B. However, portion 506c extends into portion 506e via portion 506d at an acute convex curve with a relatively small radius of curvature, such that may approximate a fold. Portion 506e may then extend at a relatively wide radius of curvature (e.g., wider than, more than twice, more than 5 times, more than 7 times, or more than 10 times that of portion 506d) into saddle region 504.

The retention member of FIG. 5C may be seen to have generally reflective types of changes of curvature about plane ‘B′,’ which may be orthogonal to axis A-A as described above. For example, while a first side of the retention member comprises, beginning at saddle region 502, a concave segment (portion 506a) followed by a convex segment (portion 506b) followed by a straight segment (portion 506c), a second side of the retention member comprises, beginning at saddle region 504, a concave segment (portion 506e) followed by a convex segment (portion 506d) followed by a straight segment (portion 506e). Accordingly, the two sides of the retention member may be configured to resist migration and/or dislodgement in reflective directions to each other. For example, the first side may more strongly resist migration and/or dislodgement along direction A and the second side may more strongly resist migration and/or dislodgement along direction B than the respective other.

However, the geometry of curves between the two sides of the retention member about plane B′ may be seen to differ, for example, in radii of curvatures of respective portions. Without wishing to be bound by any particular theory, it is believed that the more acute interior angle of portion 506d as opposed to portion 506b may allow it to latch into and/or onto apposed or engaged tissue, increase a friction against apposed or engaged tissue, and/or facilitate a component of a resistive force as discussed above along a longer projection along axis A-A, which may further improve its respective resistance to migration and/or dislodgement. Accordingly, the retention member of FIG. 5C may thus be configured to asymmetrically resist migration and/or dislodgement between its sides.

Configurations of retention members with asymmetric resistance may enable customized engagement of stents with apposed or engaged tissue based on the needs and/or mechanical behavior of the tissue or a target tissue. For example, the saddle region 504 of the example of FIG. 5C may be positioned along stricture S as described above in order to take advantage of a lower risk of the retention member to allow stent migration along arrow B than along arrow A based on its asymmetric sides. It is further contemplated that portions 506b and 506d may present asymmetrically gripping tissue interfaces, which may be chosen based on an ability and/or sensitivity of respectively apposed or engaged tissue to withstand such a grip. For example, a retention member such as that of FIG. 5C may be arranged such that saddle region 502 rather than saddle region 504 is placed across an inflamed region or other sensitive target area based on a lower risk of an interface with portion 506b to further inflame or irritate the tissue than an interface with portion 506d. Embodiments are not limited in this context.

Turning to FIG. 5D, an example of a retention member may include various straight portions transverse to axis A-A as discussed above. Saddle region 502 extends through portion 506a in an acute interior angle into portion 506b, which is thus bent back over a surface of saddle region 502. Portion 506b continues through portion 506c in an acute interior angle into portion 506d extending over and therebeyond, which may be substantially parallel to saddle region 502 and/or saddle region 504, and which may correspond to one or more of cylindrical regions 268, 270. Portion 506d extends through portion 506e at an acute interior angle into portion 506f, which thus bends back under at least part of portion 506d before wrapping back in an acute interior angle through portion 506g into saddle region 504, which additionally extends below and therebeyond portion 506f.

In some embodiments, angles formed by tangent lines of saddle region 502 and portion 506b and by tangent lines of saddle region 504 and portion 506f may be the same, and/or angles formed by tangent lines of portion 506b and portion 506d and by tangent lines of portion 506d and portion 506f may have the same magnitude (tangent lines and angles not illustrated for the sake of simplicity in the drawings). In some embodiments, each of the angles formed by tangent lines of saddle region 502 and portion 506b, by tangent lines of saddle region 504 and portion 506f, by tangent lines of portion 506b and portion 506d, and by tangent lines of portion 506d and portion 506f may have the same or substantially the same magnitude, which may be, for example, at least 45 degrees and less than 90 degrees.

While the example of FIG. 5D is illustrated with portions 506a, 506c, 506e, and 506d as comprising small enough radii of curvature such as to render the retention member as approximately trapezoidal in shape (i.e., with distinct corner regions at portions 506a, 506c, 506e, and 506d), alternative embodiments may include larger radii of curvature at all or any of portions 506a, 506c, 506e, and 506d. For example, one will recognize that increasing the radii of curvature of portions 506a, 506c, 506e, and 506d in the example of FIG. 5D may render the retention member as having an hourglass shape. Embodiments are not limited in this context.

The example of FIG. 5D contains reflective types of changes of curves about plane B′ as discussed above with respect to FIG. 5C. However, the example of FIG. 5D further comprises reflective geometries of portions about plane B′. Accordingly, each of the sides of the retention member of FIG. 5D may be configured to equally resist a correspondingly reflective force without migrating and/or dislodging.

FIG. 5E contains an additional example similar to that of FIG. 5D, but in which intervening straight segments are not located between concave and convex portions. Rather, saddle region 502 extends into portion 506a, which bends concavely into portion 506b. Portion 506b continues convexly into portion 506c, which may correspond with one of cylindrical regions 268, 270 as discussed above. Portion 506c extends into portion 506d, which bends convexly into portion 506f. Saddle region 504 extends from portion 506f. Whereas the example illustrated in FIG. 5D may thus be seen to have sides which are approximately “Z” shaped, the example of FIG. 5E may thus have sides which are approximately “S” shaped.

While the example of FIG. 5D described interior angles defined by tangent lines of adjacent straight portions as being at least 45 degrees and less than 90 degrees, the example of FIG. 5E may include interior angles defined by any or each of portions 506a, 506b, 506d, or 506e having virtually any magnitude. For example, the curves of the illustrated portions 506a, 506b, 506d, and 506e may be seen to define angles of approximately 0 degrees, as they each wrap, curl, or fold back so as to extend in substantially the same directions at either of their respective ends. In alternative examples (not shown), segments such as portions 506a, 506b, 506d, and/or 506e may comprise larger interior angles (e.g., 0 to 90 degrees) so as to extend in opposing transverse directions at either of their respective ends.

In some examples (not shown), segments such as portions 506a, 506b, 506d, and/or 506e may wrap, curl, or fold back so as to extend back at each end in intersecting directions (e.g., in a greater-than-half circle). Accordingly, the portions 506b, 506d may begin with a diameter less than a maximum diameter of respective portions 506a, 506e (e.g., they may sag downwards over or form overhangs over respective saddle regions 502, 504).

Various examples (not shown) may include curved portions such as described with respect to FIG. 5E but have intervening straight portions such as described with respect to FIG. 5D. Embodiments are not limited in this context.

It will be recognized that a wrap, curl, or fold of a curved portion back over itself may contribute to a higher resistance of the respective portion to straightening, for example, due to the greater force necessary to overcome an internal opposing force within the portion so as to be able to change a direction of an end and thereby longitudinally extend the portion into a stretched configuration. Accordingly, the example of FIG. 5F includes portion 506e which folds over itself to create a projection extending over saddle region 504, with portion 506f extending therebetween. Additionally, in the example of FIG. 5F, saddle region 502 extends into portion 506b through portion 506a at an approximately right angle, and portion 506b extends into portion 506d through portion 506c at approximately a right angle. Portion 506c then continues into portion 506e discussed above.

The example of FIG. 5G additionally includes an overhanging segment which extends over a saddle region, but which is formed by a portion defining an interior angle of slightly greater than 0 degrees. In particular, saddle region 502 extends into portion 506a, which bends concavely to the beginning of portion 506b, thereby defining an interior angle between 0 degrees and 90 degrees. Portion 506b thus extends radially outward from over portion 506a, saddle region 502, or both, bending back convexly over itself to meet portion 506c. Portion 506c may then extend to portion 506d. Portion 506c may, in various examples, be one of cylindrical regions 268, 270 as discussed above, and in some examples may be substantially parallel to saddle region 502, saddle region 504, or both. Portion 506d defines an obtuse angle extending between portion 506c and portion 506e. Portion 506e thus slopes towards saddle region 504, to which it is joined by portion 506f at a concave obtuse angle. Without wishing to be bound by any particular theory, it is believed that the relatively larger radii of curvature and/or the smaller angular changes between respective portions of the example of FIG. 5G as opposed to those of the example of FIG. 5F may result in a lower pull-out force, a tissue interface which more closely conforms to a shape of apposed or engaged tissue, or both.

With respect to FIG. 5H, the saddle region 502 may extend into a concave surface of portion 506a, which may extend into portion 506b. The portion 506b comprises a straight edge which extends at an angle of less than 90 degrees back over saddle region 502. In some embodiments, an inward wall surface may extend a distance of 0.0-5.0 mm along a longitudinal axis of the cylindrical saddle region towards a vertical center plane thereof. For example, portion 506b may extend 0.5-1.5 mm, 0.2-2.0 mm, or the like over saddle region 502. However, in other embodiments in line with the present disclosure, a portion of a retention member may extend a greater distance over a saddle region as described herein. The portion 506c comprises a convex curve, which extends between the portion 506b and the portion 506d. The portion 506d may comprise a portion parallel to a surface of the saddle region 502, and may correspond to one of cylindrical regions 268, 270 as discussed above. The portion 506d extends into portion 506e, which may comprise a convex surface. In various embodiments, an interior radius of curvature defined by portion 506e may be greater than an interior radius of curvature defined by portion 506c. However, it will be recognized that alternative embodiments may include varying relative radii of circumferences between portions. One or more convex and/or concave surfaces as described herein may include the same or various angles of completion of curvature, radii of circumference, lengths, other feature, or any combination thereof.

Regarding the example embodiment illustrated in FIG. SI, a retention member may comprise a similar saddle region 502 and portions 506a, 506b as described with respect to FIG. 5H. Portion 506b may similarly extend into portion 506c. However, portion 506c as illustrated in FIG. SI may have a greater angle of completion of curvature than as illustrated in FIG. 5H, for example, such that it extends directly into portion 506d. Portion 506d may comprise a concave surface which joins portion 506c to saddle region 504. In some embodiments, portion 506c or other segment comprising one or more portions may comprise varying radii of curvature along its length, such as a non-uniform and/or otherwise undulating curve.

FIG. 5J illustrates another exemplary embodiment, in which a retention member may comprise a similar saddle region 502 and portions 506a, 506b, 506c, 506d as described with respect to FIG. 5H. However, portion 506e extending from portion 506d as illustrated in FIG. 5J may comprise a larger radius of curvature than as illustrated in FIG. 5H. In FIG. 5J, portion 506f extending from portion 506e may comprise a straight edge extending from portion 506e back towards the saddle region 502. The portion 506f may extend into a portion 506g, which is illustrated in FIG. 5J as comprising a concave portion. Portion 506c may extend into the saddle region 504. In some embodiments, an angle defined by portion 506f and saddle region 504, or an angle of portion 506g, may be less than 90 degrees.

An additional exemplary embodiment is illustrated in FIG. 5K. In FIG. 5K, the portion 506a may comprise a concave curve which extends into a straight edge of portion 506b. The portion 506b may extend into a convex curve of portion 506c, which may in turn extend into portion 506d. Portion 506d may correspond to one of cylindrical regions 268, 270 as described above. The portion 506d may extend into the portion 506e, which may comprise a convex curve. The portion 506e may in turn extend into the portion 506f, which may comprise a concave segment. The portion 506f may extend into the saddle region 504. However, at least one of portions 506e, 506f may comprise smaller radii of curvature, e.g., as illustrated with respect to the portions 506e, 506f as illustrated in FIG. 5H. In some embodiments, a concave or convex curve of at least one portion may comprise a tight enough radius of curvature to comprise a crease or other fold. Without wishing to be bound by any particular theory, embodiments comprising retention member configurations as illustrated in FIG. 5K may thus have a greater pull out force, a more stable interaction with apposed or engaged tissue along at least portion 506e, or both, than embodiments comprising retention member configurations as illustrated in FIG. 5H.

Regarding the example of FIG. 5L, a retention member may comprise various similar portions as described with respect to at least one of FIGS. 5A-5K. For example, portions 506d and 506e may respectively comprise one or more similarities to portions 506b and 506a as described with respect to FIG. 5F, with the exception that portion 506e of FIG. 5L extends into saddle region 504 whereas portion 506a of FIG. 5F extends into saddle region 502. In at least the example illustrated in FIG. 5L, portion 506a continuing from saddle region 502 may extend, or dip, into a narrower diameter than saddle region 502. Portion 506b may extend from portion 506a and bend back to have a larger diameter than saddle region 502. In various examples, portion 506b may extend back over one or both of portion 506a and at least part of saddle region 502. Portion 506c extends from portion 506b and bends convexly to meet portion 506d as described above. Various portions as described herein may comprise a small enough radius of curvature as to constitute a right angle or a relatively right angle. For example, a portion, such as portion 506e as illustrated in FIG. 5L, may comprise a radius of curvature of 0.0-0.5 mm, for example, 0.1-0.3 mm.

Referring now to the example of FIG. 5M, retention members may comprise two or more parallel surfaces. For example, portion 506b extending from saddle region 502 through portion 506a and portion 506f extending from saddle region 504 through portion 506g may comprise parallel or substantially parallel surfaces. Portion 506d may extend between portions 506b and 506f, connected to portion 506b via portion 506c and connected to portion 506f via portion 506e. Portion 506d may, in some examples, correspond to one of cylindrical regions 268, 270 as described above.

While portions 506b, 506f are illustrated in FIG. 5M as extending substantially perpendicularly from one or both of saddle regions 502, 504 (e.g., substantially perpendicularly from axis A-A as discussed above), surfaces of retention members according to the present disclosure may be otherwise transverse to one or more of saddle region 502, 504 or axis A-A. For example, rather than each defining interior angles of 90 degrees as illustrated, portions 506a and 506g may be otherwise supplementary. For example, portion 506a may define an interior angle of 30 degrees and portion 506g may define an interior angle of 150 degrees, portion 506a may define an interior angle of 45 degrees and portion 506g may define an interior angle of 135 degrees, portion 506a may define an interior angle of 50 degrees and portion 506g may define an interior angle of 130 degrees, or the like. In other embodiments, portions 506a and 506g may not be complementary, such as portions 506b and 506e as described with respect to FIG. 5L. Any number of portions of stent wall may be used to then connect respective portions such that a retention member is formed, which may in some embodiments form a complete wall or division between a lumen (e.g., lumen 122) and an exterior of the stent. Embodiments are not limited in this context. Embodiments are not limited in this context.

While various portions are described with respect to FIGS. 5A-5M, it will be appreciated that various embodiments may comprise one or more similarities and/or differences from the illustrated examples. For example, retention members may comprise more or fewer portions than described above, and any portion thereof may comprise at least one concave section, convex section, straight edge, or any combination thereof. A portion may extend toward a first or second end of the device, perpendicular to a surface of a cylindrical saddle region, parallel to a surface of a cylindrical saddle region, at another angle with respect to a surface of a cylindrical saddle region, or any combination thereof. Furthermore, dimensions and/or orientations of any of the portions described with respect to FIGS. 5A-5M may be applied to other portions described therewith, alternatively or in combination, or with portions and/or retention member profiles otherwise with the scope of the present disclosure. Additionally, or alternatively, a profile of a retention member may vary rotationally about an axis. For example, while not illustrated for the sake of simplicity, a retention member may comprise an asymmetrical cross section in longitudinal plane A′ about axis A-A as described above. For example, a cross section of a retention member when viewed above axis A-A within plane A′ may comprise a shape as described with respect to FIG. 5A, but when viewed below axis A-A within plane A′ may comprise a shape as described with respect to FIG. 5B. Alternatively, or additionally, any combination of cross-sectional shapes or features as described above with respect to FIGS. 5A-5M are presently contemplated. Embodiments are not limited in this context.

In several embodiments, smaller radii of curvature in portions may contribute to a higher retentive strength of the corresponding retention member. For example, the configuration illustrated in FIG. 51 may comprise a greater resistance to a deformation than the respective configuration of FIG. 5M. Accordingly, embodiments may be configured in accordance with various retention member strength requirements as necessitated by at least one particular procedure, tissue, or other consideration.

It is additionally contemplated that various retention member configurations as described with respect to FIGS. 5A-5M may provide additional benefits over conventional devices. For example, various retention member configurations may create a space between apposed or engaged tissue and a stent saddle region. For example, retention members with configurations such as those of FIGS. 5D-5L may, by virtue of their overlapping portions, hold apposed or engaged tissue away from at least a part of one or both of saddle regions 502, 504. Accordingly, the respective retention members may be configured to reduce contact of one or both of saddle regions 502, 504 with at least one target area along an apposed or engaged tissue, which may thereby reduce a risk of inflammation of the at least one target area due to an interface thereof with a stent.

Various stents according to the present disclosure may comprise multiple retention members as described with respect to any of FIGS. 5A-5M, which may be configured to hold apposed or engaged tissue entirely apart from a saddle region extending therebetween. For example, retention members 110, 112 as described above may each comprise any of the configurations as described with respect to FIGS. 5A-5M and may be configured to hold an apposed or engaged tissue entirely away from middle region 108. Retention members may be configured to hold apposed or engaged tissue at least 1 mm, 2 mm, 3 mm, 4 mm, or 5 mm away from a saddle region, although distances less than 1 mm, greater than 5 mm, and any intermediate distance are additionally presently contemplated. In some embodiments, retention members may be configured to hold a therapeutic agent, such as a growth medium, sealant, tissue scaffold, anti-inflammatory agent, adhesive agent, or the like, along a saddle region and between the retention members such that apposed or engaged tissue is exposed to the therapeutic agent. For example, retention members 110, 112 may be configured to hold an apposed or engaged tissue apart from middle region 108 such that a space is defined between them, which may be preloaded or subsequently filled with a therapeutic agent, for example, via an injection needle extended through tissue or through a wall of the stent. Embodiments are not limited in this context.

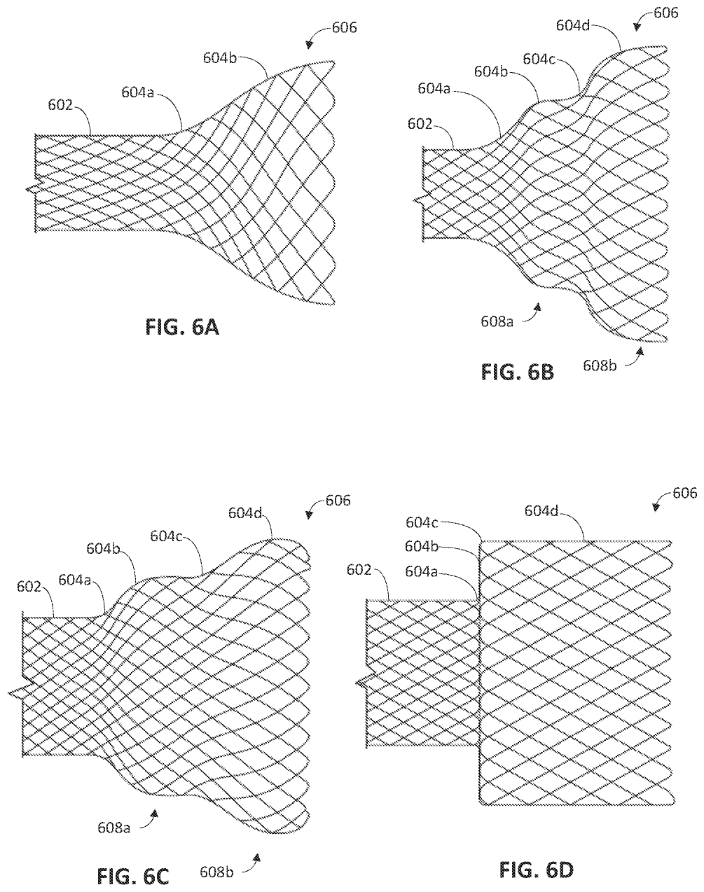

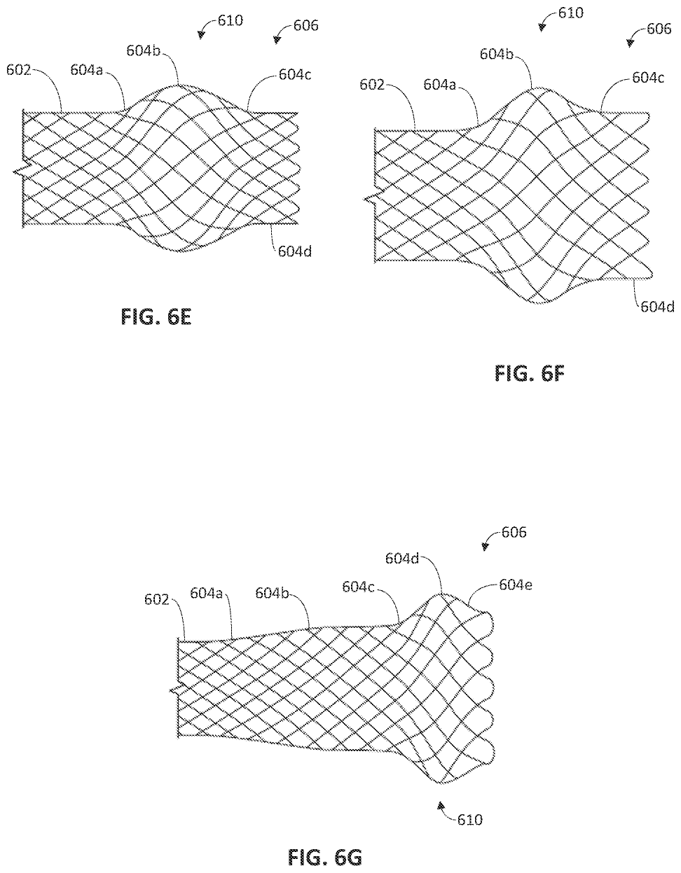

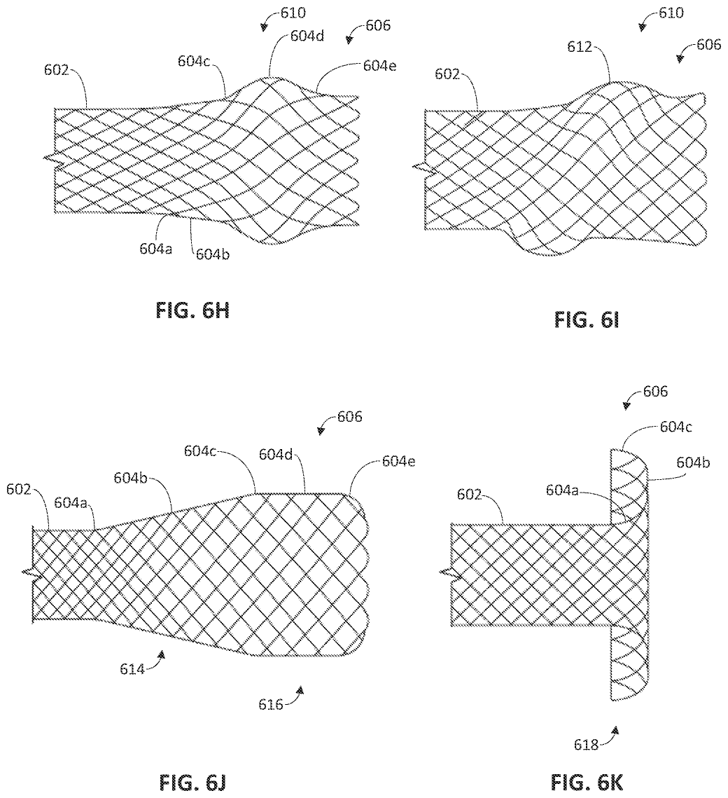

FIGS. 6A-6K illustrate side views of additional examples of retention members which may be found in stents according to the present disclosure, such as any of retention members 114, 116, 214, 216, 314, and 316. However, retention members configured as described with respect to FIGS. 6A-6K may comprise a saddle region 602 extending only from one side. For example, saddle region 602 may correspond to one of saddle regions 118, 120, 318, 320, 502, or 504 as discussed above. Each of FIGS. 6A-6K further includes various portions 604 defined between stent wall inflection points (such as n1-5 as described above, not shown in FIGS. 6A-6K for the sake of simplicity). For the sake of simplicity, subsequently described portions 604 of FIGS. 6A-6K will be referred to as 604a, 604b, 604c, 604d, and the like. However, it will be appreciated that any number and/or configuration of portions 604 may be included in retention member configurations according to the present disclosure.

Various examples of retention members may comprise a flared end 606 having an end diameter greater than that of saddle region 602. For example, in FIG. 6A, portion 604a extending from saddle region 602 bends concavely outwards to have a wider diameter than saddle region 602, and portion 604b extends from portion 604a to further increase in diameter. Accordingly, portions 604a, 604b in FIG. 6A form a continuously flared end 606.

Various embodiments may alternatively include at least one flared end having a stepped, undulating, or otherwise varying configuration. For example, in FIG. 6B, portion 604a continues concavely from saddle region 602 into portion 604b, and portion 604b bends convexly to form a first step 608a. From portion 604b, portion 604c bends concavely outward to portion 604d, which bends convexly to form a second step 608b. Retention members may include any number of steps, such as 1, 2, 3, 4, 5, or more. While not illustrated for the sake of brevity of the drawings, steps or other surface features as described herein may include one or more straight portions, for example, between adjacent concave and convex portions, or intersecting concave or convex portions as described herein.

While the example retention member of FIG. 6B can be seen to consistently increase in diameter along its length, various embodiments may include retention members which include at least one section of a retention member with a decreasing diameter. For example, in FIG. 6C, portion 604a extends from saddle region 602 and together with portion 604b forms first step 608a as described with respect to FIG. 6B. Similarly, portion 604c extends from portion 604b and together with portion 604d forms second step 608b as described with respect to FIG. 6B. However, each of portions 604b and 604d can be seen to define a smaller interior angle than in FIG. 6B, thereby decreasing in diameter along their length. Without wishing to be bound by any particular theory, it is believed that the inward curling of each of these step ends may contribute to a reduced risk of traumatic interference with apposed or engaged tissue and/or more closely approximate an apposed or engaged tissue shape.

The example retention member of FIG. 6D includes a portion 604a which extends from saddle region 602 to portion 604b. Portion 604a as illustrated in FIG. 6D comprises a concave angle of approximately 90 degrees such that portion 604b extends approximately perpendicularly from axis A-A as described above. However, 604a may comprise alternative interior angles such that portion 604b is otherwise transversely oriented with respect to axis A-A and/or saddle region 602. Portion 604c connects portion 604b to portion 604d at a second angle. While the interior angle of portion 604c is illustrated as approximately 90 degrees, any angle may be defined by portion 604c. In various examples, angles defined by portions 604a and 604c may be supplementary to each other, for example, such that saddle region 602 and portion 604d extending from portion 604c may be substantially parallel to one another.

Furthermore, while the radii of curvature of portions 604a, 604c are illustrated in FIG. 6D as substantially small enough to constitute a fold or a crease, for example, 0.0-0.5 mm, such as 0.1-0.3 mm, larger radii of curvature are presently contemplated, for example, 0.5-5.0 mm or larger. Embodiments are not limited in this context.

FIG. 6E-6I illustrate retention members which include a bulge 610 extending circumferentially about a stent. Turning to FIGS. 6E-6F, portion 604a extends from saddle region 602 and concavely bends to portion 604b. Portion 604b bends convexly to define a maximum diameter of bulge 610 (diameter not illustrated for the sake of simplicity in the drawings). From portion 604b, portion 604c bends concavely to extend to portion 604d at an end 606 of the stent. While the example of FIG. 6E comprises a portion 604d and/or end 606 having a diameter substantially the same as a diameter of saddle region 602, FIG. 6F comprises a portion 604d and/or end 606 having a diameter larger than a diameter of saddle region 602, for example, 10%, 20%, 30%, 40%, 50%, or 60% larger than the diameter of saddle region 602 (diameters not illustrated for the sake of simplicity in the drawings). In other embodiments, retention members may comprise ends such as end 606 having diameters less than a diameter of an attached saddle region, for example, 10%, 20%, 30%, 40%, 50%, or 60% smaller than the diameter of the corresponding saddle region. Embodiments are not limited in this context.

In FIG. 6G, as opposed to FIG. 6F, bulge 610 is located at an end of a stent region of increased diameter rather than at a beginning thereof. For example, in FIG. 6G, portion 604a bends from saddle region 602 outward to a portion 604b having a larger diameter than saddle region 602. Portion 604c then bends concavely further outward to portion 604d, which then defines a maximum diameter of bulge 610. Portion 604e may optionally bend back radially inward to have a diameter less than the maximum diameter of bulge 610, for example, greater than or substantially equal to the maximum diameter of 604b.

Without wishing to be bound by any particular theory, it is believed that retention members defined by portions with larger radii of curvature may comprise gentler tissue interfaces and/or interfaces which more closely approximate a natural tissue surface than retention members defined by portions with corresponding smaller radii of curvature. For example, FIG. 6H illustrates an example of a retention member having various similar features as described with respect to FIG. 6G, but in which each curved portion comprises a correspondingly larger radius of curvature than in the example of FIG. 6G. Accordingly, the example of FIG. 6H may comprise an increased correspondence to an apposed or engaged tissue's natural shape than the example of FIG. 6G, the example of FIG. 6G may more securely lodge within a tissue lumen than the example of FIG. 6H, or both.

While examples of FIGS. 6E-6H comprise only a single bulge each, embodiments are additionally contemplated which comprise multiple bulges. For example, multiple bulges 610 may circumferentially extend about a stent (not shown). Alternatively, or additionally, a bulge may wrap helically about a stent, as is illustrated by bulge 612 of FIG. 61. In other embodiments, one or more bulges may not extend fully about a circumference of a stent but may instead extend asymmetrically about part of a circumference of a stent. Embodiments are not limited in this context.

In the example of FIG. 6J, portion 604a connects saddle region 602 to a portion 604b comprising a straight edge extending radially outward from saddle region 602 and thereby forming a ramped portion 614. Portion 604b continues into portion 604c, which bends into portion 604d. Portion 604d comprises a second straight edge, which may extend parallel with saddle region 602 and thereby form a cylindrical region 616 of the stent. Ramped portion 614 and/or cylindrical portion 616 may each individually or together comprise a longitudinal length at least one fourth, one third, one half, two thirds, three fourths, or the same length as a length of saddle region 602.

Optionally, portion 604e may then continue from portion 604d, decreasing in diameter at end 606 of the stent. Without wishing to be bound by any particular theory, it is believed that a reduction in diameter of a stent at its end may reduce a risk of the stent to traumatically engage with tissue at or along said end.

FIG. 6K illustrates an example of a retention member comprising an overlap region 618, which may be formed by part of a stent bending back over itself. For example, portion 604a extends from saddle region 602 into portion 604b, which extends radially outward from saddle region 602. While portion 604b is illustrated as being substantially orthogonal to a surface of saddle region 602, portion 604b may be set at an otherwise transverse angle to saddle region 602. Portion 604c may optionally extend from portion 604b so as to overlap at least part of portion 604b, portion 604a, saddle region 602, or any combination thereof.

In various embodiments, overlap region 618 may thus present an atraumatic interface at end 606 of a stent, a cupped or closed end to a gap defined along a surface of saddle region 602, or both. Embodiments are not limited in this context.

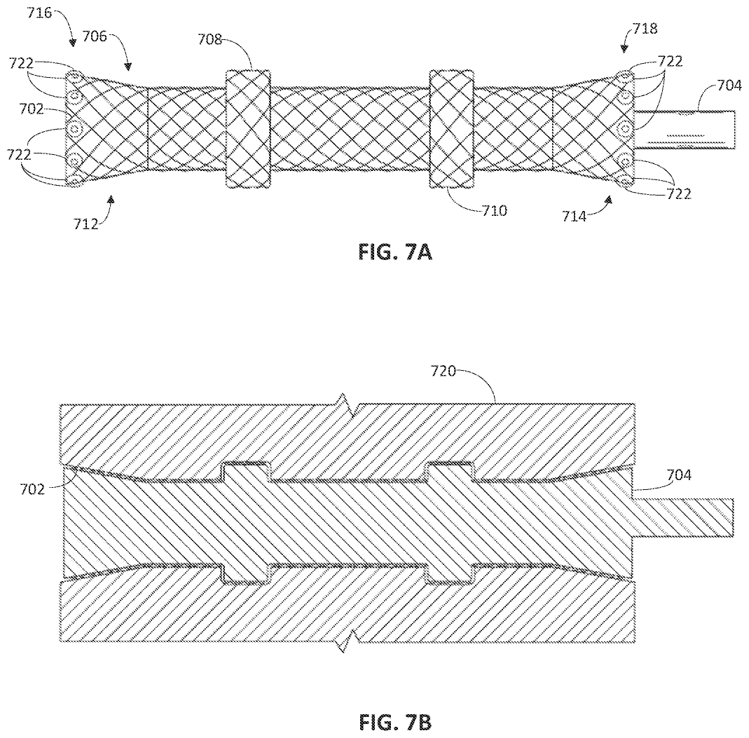

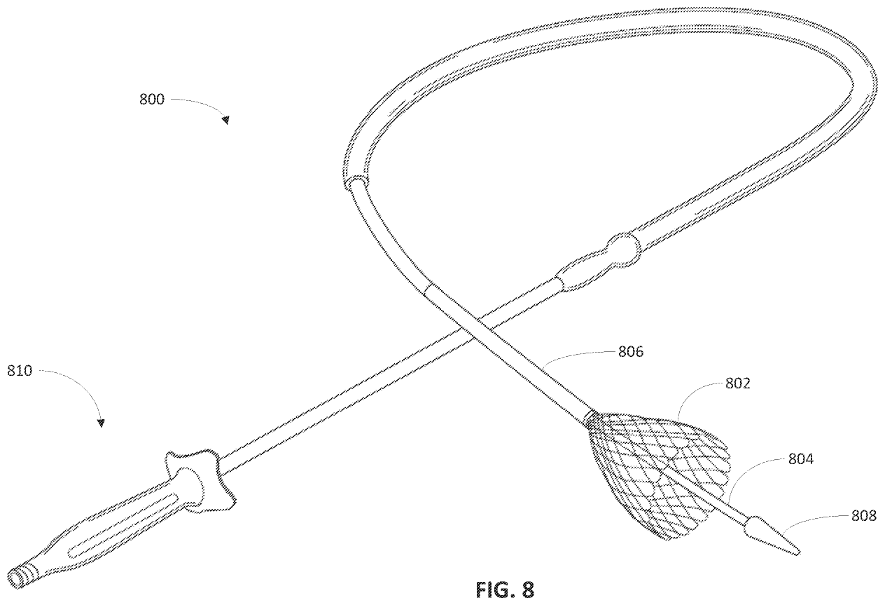

Stents according to the present disclosure may be performed with one or more retention members as described herein, for example, by wrapping at least one filament braid 702 about a mandrel 704 to form stent 706, as illustrated in FIG. 7A. In various embodiments, a preformed configuration as described herein may comprise a deployed configuration, expanded configuration, unconstrained configuration, or the like.

Mandrel 704 may comprise various washers, extensions, or other protrusions, such as washers 708 and 710 and ramped protrusions 712 and 714, about which the at least one filament braid 702 may be disposed and set, for example, by heat setting, to form correspondingly shaped retention members. For example, any retention member as described with respect to FIGS. 5A-5M and FIGS. 6A-6K may be formed by setting the shape of a filament braid about a correspondingly shaped protrusion of a mandrel. In various examples, mandrel 704 may comprise one or more grooves into which the at least one filament braid 702 may be aligned (not shown). In various embodiments, the at least one filament braid 702 may be wrapped around at least one projection such as screws 722 to form an end 716 and/or end 718 of a stent with an edge of curved filament, such as end 606 as illustrated in FIGS. 6A-6K.

As is shown in the cross-sectional view of FIG. 7B, one or more clamps 720 may be used to constrain the at least one filament braid 702 against the surface of mandrel 704, for example, during a heat setting process. Accordingly, stent 706 may be formed with precise dimensions including diameters, widths, radii of curvature, or the like according to a desired configuration. Clamps 720 may include one or more components which may be clamped around mandrel 704, slid over an end of mandrel 704, or otherwise constrained about mandrel 704.

It will be appreciated that various stents according to the present disclosure may be pre-formed, for example, around mandrel 704, with one or more weave pitches, braid patterns, aperture sizes, aperture geometries, or the like as described above with respect to FIGS. 3-4B. For example, the at least one filament braid 702 may be wrapped around one or more of washers 708 and 710 and ramped protrusions 712 and 714 with a different weave pitch than along another portion of mandrel 704. Embodiments are not limited in this context.

Various stents according to the present disclosure may be configured to transition between first and second configurations, such as delivery and deployed configurations, constrained and expanded configurations, or the like. In various embodiments, stents according to the disclosure may be loaded into a delivery system in the first configuration (e.g., delivery or constrained configurations) and delivered to a target location, for example, in or across one or more body tissues or lumens.

FIG. 8 illustrates an example of a delivery system 800 according to the present disclosure. A stent 802, which may be or include one or more similarities with any of the stents discussed herein, may be constrained between an inner member 804 and an outer sheath 806, wherein the outer sheath 806 is slidably disposed about the inner member 804, the inner member 804 is slidably disposed within the outer sheath 806, or both.

In some embodiments, a tip 808 may be disposed at a distal end of inner member 804. Tip 808 may be a tissue penetrating tip, and may comprise one or more of a knife, electrocautery tip, or other cutting element.

A handle 810 may be disposed at the proximal end of one or both of the inner member 804 and outer sheath 806. Handle 810 may be configured to effect of displacement of one or both of inner member 804 and outer sheath 806 with respect to each other.

The delivery system 800 may be typically used in coordination with an endoscope, a non-limiting example of which is an endoscopic ultrasound (EUS) scope (not shown). For example, delivery system 800 may be extended through a working channel of an endoscope such that handle 810 is accessible near a handle of the endoscope and tip 808 extends through the working channel of the endoscope, for example, into a patient's body cavity.

FIGS. 9A-9E illustrate use of delivery system 800 to deliver stent 802 to a target site and to deploy stent 802 at the target site. It will be understood that, while delivery of stent 802 is illustrated within a single body lumen in FIGS. 9A-9E, deliveries to alternative target sites are presently contemplated, including end-to-end anastomoses, end-to-side anastomoses, or the like. Embodiments are not limited in this context.

In FIG. 9A, tip 808 and distal ends of inner member 804 and outer sheath 806 are extended into a body lumen 900 defined by tissue 902 such that stent 802 in a delivery configuration is positioned within body lumen 900. Delivery system 800 may be positioned within body lumen 900 such that tip 808, inner member 804, and/or outer sheath 806 is positioned with respect to (e.g., a predetermined distance away from) a target location. In various embodiments, delivery system 800 or aspects thereof may be advanced until one or more markers 904 along outer sheath 806 (e.g., marker 904a, 904b, 904c (not illustrated), or the like) is aligned with a first position “P 1” within body lumen 900, corresponding to a desired location for an end of stent 802 to be positioned once deployed. Markers 904 may comprise at least one colored, echogenic, radiopaque, or otherwise visualizable material which may extend partially or completely about a circumference of outer sheath 806. In some embodiments, markers 904 may be alternatively positioned along inner member 804, or one or more additional markers may be additionally positioned along inner member 804.

When tip 808, inner member 804, and/or outer sheath 806 is aligned with position P1, outer sheath 806 may be proximally retracted with respect to inner member 804, inner member 804 may be distally extended with respect to outer sheath 806, or both such as to release a distal end 906 of stent 802 from between inner member 804 and outer sheath 806. Distal end 906 of stent 802 may correspond to all or part of a stent end as discussed above, such as first end 104 or second end 106.

As illustrated in FIG. 9B, when released from between inner member 804 and outer sheath 806, distal end 906 of stent 802 may deploy to expand into a first retention member 908, which may comprise any configuration as discussed herein (e.g., a retention member as illustrated in and/or described with respect to one of FIGS. 6A-6K). In some embodiments, first retention member 908 may expand to have a same or greater diameter than lumen 900. For example, first retention member 908 may distend tissue 902 so as to increase a diameter of lumen 900.

Inner member 804 and outer sheath 806 may be fixed with respect to each other (e.g., locked via handle 810 as discussed with respect to FIG. 8) after deployment of first retention member 908 such that delivery system 800 may be further positioned within body lumen 900. For example, delivery system 800 may be positioned within body lumen 900 such that stent 802 may be further partially deployed to form a second retention member 910 at a second reference position “P2” within body lumen 900 and/or along tissue 902. Second retention member 910 may comprise a configuration as discussed herein, such as that of a retention member as illustrated in and/or discussed with respect to any of FIGS. 5A-5M.

Positioning of delivery system 800 with respect to P2 may be determined based on visualization of a marker as discussed above at or near P2 prior to deployment of second retention member 910. For example, positioning of marker 904b at P2 may indicate that delivery system 800 is positioned within body lumen 900 such that deployment of second retention member 910 will result in second retention member 910 being positioned at P2. Marker 904b may be disposed along inner member 804 and/or outer sheath 806. For example, markers 904 along each of inner member 804 and outer sheath 806 may be aligned with each other based on a sliding of inner member 804 and outer sheath 806 with respect to each other, and the aligned markers 904 may be positioned with respect to P2. Embodiments are not limited in this context.

Once delivery system 800 is positioned such that a deployed second retention member 910 will be positioned properly within lumen 900, inner member 804 and outer sheath 806 may be unfixed with respect to each other. As illustrated in FIG. 9C, outer sheath 806 may then be further retracted with respect to inner member 804, inner member 804 may then be further extended with respect to outer sheath 806, or both, so as to continue to release an extent of stent 802 from between inner member 804 and outer sheath 806. Stent 802 may thereby be partially deployed to form second retention member 910 at the desired location.

In some examples, an intervening extent of stent body may be deployed between first retention member 908 and second retention member 910, such as cylindrical saddle region 118 as discussed above. One or both of second retention member 910 and the intervening extent of stent body may expand to have a same or greater diameter than lumen 900.

FIG. 9D illustrates further retraction of outer sheath 806 with respect to inner member 804, extension of inner member 804 with respect to outer sheath 806, or both so as to further partially deploy stent 802 to form a third retention member 912, for example, at a third reference position “P3” based on a positioning of at least one marker (not shown) at P3 prior to deployment of third retention member 912. In some embodiments, inner member 804 and outer sheath 806 may be fixed with respect to each other after deployment of second retention member 910 and unfixed with respect to each other based on positioning of at least one aspect of delivery system 800 at P3. Third retention member 912 may be deployed based on the unfixing of inner member 804 and outer sheath 806 with respect to each other. In some embodiments, a saddle region such as middle region 108, 208, 308 may be deployed between second retention member 910 and third retention member 912. One or both of the saddle region and third retention member 912 may comprise a diameter at least as great as that of lumen 900. Third retention member 912 may comprise a configuration as discussed herein, such as that of a retention member as illustrated in and/or discussed with respect to any of FIGS. 5A-5M.

As shown in FIG. 9E, after deployment of third retention member 912, outer sheath 806 may be further retracted with respect to inner member 804, inner member 804 may be further extended with respect to outer sheath 806, or both so as to completely release and thereby deploy stent 802 from between inner member 804 and outer sheath 806. Accordingly, a proximal end 916 may expand into fourth retention member 914. Similarly, as discussed above with respect to deployment of other retention members, inner member 804 and outer sheath 806 may be fixed with respect to each other and delivery system 800 positioned such that deployment of fourth retention member 914 will occur at a fourth reference position “P4,” at which point inner member 804 and outer sheath 806 may be unfixed with respect to each other. One or more markers (not shown) may be used to position the delivery system 800 accordingly.

Fourth retention member 914 may comprise any configuration as discussed herein, for example, a configuration as illustrated by and/or discussed with respect to any of FIGS. 6A-6K. A saddle region such as cylindrical saddle region 120 may be deployed between third retention member 912 and fourth retention member 914. One or both of the saddle region and fourth retention member 914 may comprise a diameter at least as great as that of lumen 900.

In various embodiments, deployment of one or more aspects of stent 802 may include a foreshortening of stent 802 (i.e., a decrease in longitudinal length thereof). For example, upon deployment, stent 802 may foreshorten up to 5%, 10%, 15%, 20%, 25%, 30%, 35%, 40%, 45%, 50%, 55%, or more, including any percentage in between. Foreshortening of stent 802 may result in one or more aspects of stent 802 aligning with respective reference or target locations within lumen 900. For example, foreshortening of stent 802 may cause first retention member 908 to be aligned with position P1, second retention member 910 to be aligned with position P2, third retention member 912 to be aligned with position P3, and/or fourth retention member 914 to be aligned with position P4. In some embodiments, stent 802 may be positioned such that aspects are respectively aligned with one or more reference positions such as P1, P2, P3, or P4 prior to complete foreshortening of stent 802. For example, first retention member 908 may be aligned with position P1 after deployment of only distal end 906 (i.e., before deployment of proximal end 916). Deployment of subsequent aspects of stent 802 (i.e., proximal end 916) may result in a sliding of deployed aspects with respect to tissue 902 and/or a repositioning of tissue 902 due to foreshortening of stent 802. For example, a friction fit of first retention member 908 with tissue 902 may cause an apposed or engaged surface of tissue 902 to distend to remain positioned along first retention member 908 as stent 802 foreshortens.

In some embodiments, foreshortening of stent 802 may be limited by opposing forces applied by apposed or engaged tissue. For example, tissue 902 may apply an opposing force to a tension applied to distal end 906 by foreshortening of stent 802 such that, when deployed in tissue, stent 802 foreshortens less than when deployed outside of tissue (e.g., in a vacuum or empty space). It will be recognized that an amount of foreshortening of a stent may then be customized based a configured interaction of the stent with apposed or engaged tissue. Accordingly, a weave pitch, braid pattern, or the like as discussed above may be customized so as to affect a radial strength of at least part of a stent and thereby an interaction of the stent with apposed or engaged tissue so as to achieve a desired foreshortening behavior of the stent when deployed in tissue.

While reference positions P1, P2, P3, and P4 are illustrated in FIGS. 9A-9E as along edges of respective retention members, it will be recognized that reference positions may alternatively, or additionally, be located along alternative edges of retention members, along a length of one or more retention members, or elsewhere. Embodiments are not limited in this context.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1. A stent, comprising:
an elongate body configured to expand between a first constrained configuration and a second unconstrained configuration,
the elongate body in the second unconstrained configuration including a first end, a second end, and a cylindrical saddle region extending therebetween, wherein:
the first end comprises a first retention member and a second retention member, and
the second end comprises a third retention member and a fourth retention member.

2. The stent of claim 16, wherein the second retention member comprises a substantially cylindrical first radially outward surface, the third retention member comprises a substantially cylindrical second radially outward surface, or both.

3. The stent of claim 17, wherein the second retention member comprises a first axially inner face joined to the first radially outward surface at a first corner, wherein the third retention member comprises a second axially inner face joined to the second radially outward surface at a second corner, or both.

4. The stent of claim 18, wherein the first axially inner face comprises a curved profile, wherein the second axially inner face comprises a curved profile, or both.

5. The stent of claim 18, wherein the first corner comprises an interior angle of 90 degrees or less, wherein the second corner comprises an interior angle of 90 degrees or less, or both.

6. The stent of claim 16, wherein the first retention member is configured to interface with tissue along an entire first longitudinal length thereof, wherein the fourth retention member is configured to interface with tissue along an entire second longitudinal length thereof, or both.

7. The stent of claim 16, wherein the first end comprises a first cylindrical portion extending between the first retention member and the second retention member, wherein the second end comprises a second cylindrical portion extending between the third retention member and the fourth retention member, or both.

8. The stent of claim 16, wherein the first and fourth retention members, the second and third retention members, or the first, second, third, and fourth retention members are each configured to atraumatically interface with tissue about a radially outwardmost circumference of the respective retention member.

9. The stent of claim 16, wherein the stent is formed from at least one braided or woven filament comprising a different pitch density, angle, or pattern at the first retention member, the second retention member, the cylindrical saddle region, the third retention member, or the fourth retention member, or any combination thereof, in the second configuration.

10. The stent of claim 16, wherein the elongate body comprises at least a partial cover.

11. A medical device, comprising:
an elongate body defining a lumen extending therethrough, the elongate body configured to transition between a first configuration and a second configuration;
wherein, in the second configuration, the elongate body comprises at least four retention features, the at least four retention features comprising at least second and third retention members each comprising a shoulder with an interior angle of 90 angles or less, each shoulder configured to interface with tissue, and the at least four retention members further comprising at least first and fourth retention members configured to atraumatically interface with tissue;
wherein, in the second configuration, the elongate body comprises a cylindrical region extending between the second and third retention members, between the first and second retention members or between the third and fourth retention members, or any combination thereof.

12. The medical device of claim 26, wherein one or more of the first, second, third, or fourth retention members are each configured to atraumatically interface with tissue about a radially outwardmost circumference of the respective retention member.

13. The medical device of claim 26, wherein the first retention member comprises an asymmetrical cross section in an orthogonal plane to a longitudinal axis extending through the lumen of the body, wherein the second retention member comprises an asymmetrical cross section in an orthogonal plane to the longitudinal axis, or both.

14. The medical device of claim 26, wherein the elongate body is formed from at least one braided or woven filament comprising a different pitch density, angle, or pattern at the first retention member, the second retention member, the cylindrical saddle region, the third retention member, or the fourth retention member, or any combination thereof, in the second configuration.

15. The medical device of claim 26, wherein the elongate body comprises at least a partial cover.

16. A system, comprising:
a delivery catheter, comprising:
an inner member; and
an outer sheath slidably disposed about the inner member, and
a stent disposed between the inner member and the outer sheath in a first configuration, the stent configured to transition between the first configuration and a second configuration when deployed from between the inner member and outer sheath, wherein, in the second configuration, the stent comprises at least four retention members comprising second and third retention members each having at least one shoulder separated by a cylindrical saddle region and first and fourth retention members disposed respectively on a side of the second and third retention members furthest from the cylindrical saddle region, each retention member configured to atraumatically interface with tissue.

17. The system of claim 31, wherein the first and fourth retention members are each configured to atraumatically interface with tissue about a radially outwardmost circumference of the respective retention member.

18. The system of claim 31, wherein the stent is formed from at least one braided or woven filament comprising a different pitch density, angle, or pattern at the first retention member, the second retention member, the cylindrical saddle region, the third retention member, or the fourth retention member, or any combination thereof, in the second configuration.

19. The system of claim 31, wherein the stent comprises at least a partial cover.

20. The system of claim 31, wherein the first retention member, the second retention member, the first retention member, or the fourth retention member, any combination thereof, comprises an asymmetrical cross section in an orthogonal plane to a longitudinal axis extending through a lumen of the stent.
